(19) United States
(12) Patent Application Publication
(10) Pub. No.: US 2005/0060100 A1
(43) Pub. Date: Mar. 17, 2005

(54) Computer software and algorithms for systems biologically linked to cellular phenotype

(75) Inventors: Perry D. Haaland, Chapel Hill, NC (US); Mohammad A. Heidaran, Cary, NC (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: Becton Dickinson and Company

(21) Appl. No.: 10/662,713

(22) Filed: Sep. 15, 2003

(51) Int. Cl. G06F 19/00; G01N 33/48; G01N 33/50; G06F 15/00; G06F 17/18; G06F 101/14; C12M 1/34; C12M 3/00; G06G 7/48; G06G 7/58

(52) U.S. Cl. 702/19; 435/287.2; 702/179; 703/11

(57) ABSTRACT

An automated method and system for identifying agents capable of eliciting a phenotypic change in a cell-type is provided. The method includes the steps of: providing receptacles in an array; and providing a statistical design including generic factor names, factor levels and experimental runs. The method further includes the steps of utilizing a software program to generate a computer representation of the statistical design by automatically mapping the identities of the agents to the generic factor names, by mapping the concentration or amounts of the agents to the factor levels and by mapping the locations of the receptacles within the array to the experimental runs. The method also includes placing different mixtures of single agents into receptacles in the array based on the computer representation of the statistical design; contacting the placed mixtures with cells; acquiring experimental data from the contacted cells; and utilizing a processor including an algorithm for comparing the acquired data with the statistical design to identify particular agent mixtures or single agents that cause a phenotypic change in the contacted cells.

### FIELD OF THE INVENTION

The present invention relates generally to the field of high throughput screening methods. In particular, the present invention relates to a computer-implemented screening method that can be used to identify mixtures of single agents and single agents within these mixtures that elicit a desired biological response in the cell.

### BACKGROUND OF THE INVENTION

For cells to be used in therapies to treat or cure diseases in humans, it is desirable to control cell fate, e.g., cell survival, proliferation and differentiation, when cells are maintained in culture in vitro. It is thus necessary to control cell surface receptor interaction with ligands. For example, in order to gain control over interactions between a cell and ligands present on the in vitro culture substrate, a suitable culture substrate, such as polystyrene, can be coated with a polymer which does not allow for cell attachment, even when serum proteins are used in the culture media. This coating thus eliminates the uncontrolled and arbitrary adsorption of the serum proteins. Biologically active ligands suitable to interact with cell surface receptors can then be immobilized on this coating while maintaining the biological activity of the ligands. This concept is known. For example, it is known to use hyaluronic acid or algenic acid as a surface coating upon which the cell adhesion ligands can be immobilized using chemistries resulting in stable covalent bonds between the coating and the cell adhesion ligands. This prevents the cell adhesion ligand from being solubilized and leaving the surface. Moreover, the coating itself does not support cell adhesion. This is described in copending, commonly owned U.S. application Ser. No. 10/259,797, filed Sep. 30, 2002.

It is likely that mixtures of agents are required in order to achieve a desired cell fate. A great number of growth effector molecules are known. These include growth factors, hormones, peptides, small molecules and extracellular binding molecules. It can thus be a tedious task to find the right growth effector or growth effector combinations to achieve a desired cell fate for a given cell type.

Therefore, there is a need in the art for higher throughput methods to identify agents useful to achieve a desired cell fate for a given cell type. This is of particular interest for cells that do not survive or only survive by drastically altering their differentiation state in conventional cell culture systems, a prime example being primary mammalian cells. In particular, there is a need in the art for a computer-implemented, statistically designed experimental method and a system for its implementation to systematically explore the interactions between mixtures of factors that are required in order to achieve a desired cell fate. Preferably, the higher throughput method would include robotic preparation of the experimental conditions and meta-analysis.

### SUMMARY OF THE INVENTION

The present invention provides an automated method and system for identifying agents that cause a phenotypic change in a cell. The method includes providing receptacles in an array and providing a statistical design including generic factor names, factor levels and experimental runs. The method further includes placing different mixtures of single agents into select ones of the receptacles according to a computer representation of the statistical design; and utilizing a software program to generate the computer representation of the design. The software automatically maps the identities of the agents to the generic factor names, maps the concentration or amounts of the agents to the factor levels, and maps the locations of the receptacles within the array to the experimental runs. Once the different mixtures have been correctly placed into receptacles in accordance with the computer representation of the design, the placed mixtures are contacted with whole cells that are capable of changing their phenotype.

The method also includes acquiring data indicative of a phenotypic change in the contacted cells and utilizing a processor including an algorithm for comparing the acquired data with the statistical design to identify which of the agent mixtures and/or which single agents are effective in causing the phenotypic change in the contacted cells. The method further includes storing the statistical design, the identities of the agents, the computer representation of the design, the acquired experimental data and the results of the algorithm comparison in one or more databases.

The present invention also provides a system for implementing the method just described. The system includes an array of receptacles, selective ones of which are for receiving (i) different mixtures of single agents; and (ii) fluid including cells. The system also includes a statistical design including generic factor names, factor levels, and experimental runs; and a software program for generating a computer representation of the design. The software program automatically maps the identities of the agents to the generic factor names, maps the concentration of or amounts of the agents to the factor levels and maps the locations of the receptacles within the array to the experimental runs. The system also includes acquired experimental data indicative of the phenotypic change in the cells; and a processor including an algorithm for comparing the experimental data with the statistical design to identify the mixtures and/or single agents which are effective in causing the phenotypic change in the cells. Further included in the system are one or more databases for storing the statistical design, the agents' identities, the computer representation of the design, the acquired experimental data, and the results of the algorithm comparison.

### BRIEF DESCRIPTION OF THE FIGURES

FIGS. 1A and 1B are flow charts of an exemplary automated process according to the present invention by which best mixtures and/or best agents capable of eliciting a phenotypic change in a cell can be identified in high throughput fashion.

FIG. 2 is a flow chart of an exemplary process according to the present invention by which a biological model can be created and/or revised using the information derived from the process in FIGS. 1A and 1B.

FIG. 3 illustrates exemplary cellular pathways hypotesized for the action of an agent mixture (M) on a biological system.

FIG. 4 is a schematic diagram of an exemplary embodiment of a computer system which can be used to carry out the method of the present invention.

FIG. 5 is a schematic diagram which illustrates a preferred embodiment of components of the system of the present invention.

FIG. 6 is a schematic representation of test wells in one embodiment of the present invention.

FIG. 7 is a schematic representation of a 96-well plate layout comprising different mixtures of single agents. In the embodiment shown, the layout is created using a statistical design in which generic factors in the design represent single agents, which are combined to form the different mixtures.

FIG. 8 is a schematic representation of a scenario that can be used in developing the statistical design of the method of the present invention.

FIG. 9 is a schematic representation of a further scenario that can be used in developing the statistical design of the method of the present invention.

FIG. 10 is a spreadsheet which is a computer representation of a mixture design. The layout shown is for a 96-well plate and was developed using the scenario in FIG. 9, wherein the total fluid volume in a well is divided up based on the number of factors present.

FIG. 11 shows a 96-well plate layout created based on a statistical design of the spreadsheet in FIG. 10.

FIG. 12 is a fluorescent microscope image of fluorescently labeled cells attached to the wells of the 96-well plate with the layout shown in FIG. 11.

FIG. 13 is a graph of the nuclei count vs. well No. obtained following analysis of the microscope image in FIG. 12.

FIG. 14 is a graph of Ln (cell count−no serum+1) vs. deviation from the reference blend obtained using a mixture-model analysis of information from FIGS. 9-13.

FIG. 15 is a graph of Ln (cell count−10% serum+1) vs. deviation from the reference blend obtained using a mixture-model analysis of information from FIGS. 9-13.

FIG. 16(a-d) is a spreadsheet showing a Plackett-Burman statistical design for the layout of a 96-well plate.

FIG. 17 shows the identity of the factors in the statistical design in FIG. 16.

### DETAILED DESCRIPTION OF THE INVENTION

As defined herein, “agents” are growth effector molecules that bind to cells and regulate the survival, differentiation, proliferation or maturation of target cells or tissue. Examples of suitable agents for use in the present invention include growth factors, extracellular matrix molecules, peptides, hormones and cytokines which can either be in solution or bound to a culture surface, such as a well surface, scaffold surface, bead surface, etc.

The term “agent-immobilizing material” is defined herein as a biocompatible polymer that can serve as a link between the culture surface and an agent.

As defined herein, the term “immobilize,” “immobilized,” and the like is to render an agent(s), i.e., growth effector molecules, immobile on a culture surface, such as a well surface or the surface of a scaffold contained within a well. This term is intended to encompass passive adsorption of the agents to the culture surface, as well as direct or indirect covalent attachment of the agents to the culture surface.

“Factors” are the names of the variables in the experiment, and represent the things that the experiment changes from one trial or run (for e.g., one well) to the next. In the present invention, “factor” is a generic name for a single agent or mixture of single agents. Factors are combined according to a statistical design to form different mixtures in the experiment.

“Statistical Design”, as defined herein is an experimental design that assists the user in finding a combination of adjustable variables (i.e., factors) to produce the best experimental outcome, dramatically reducing the number of experiments needed to achieve that objective. In the present invention, a suitable statistical design is generated using generic factor names which represent the agents being tested. The design includes factor levels that can be the amounts and/or concentrations of the factors or that can be converted to the actual amounts and/or concentrations of the factors. The design also includes experimental runs which are numbered. Experimental runs specify the combinations of factors and the levels thereof to test, and each corresponds to a single well on a multiwell plate, for example. The experimental runs can be mapped to wells on a generic multiwell plate

As used herein, the terms “pre-treatment” and “pre-treated” refers to the addition to a surface or other substrate of functional groups which are chemically involved in the covalent bond subsequently formed with the agent-immobilizing material (i.e., a biocompatible polymer). For example, a surface of a microtitre well can be subjected to arnino-plasma treatment to create an amine-rich surface onto which the agent-immobilizing material may be coupled.

The term “array,” “receptacle array,” and the like as defined herein is a plurality of unique containers, such as tubes or wells, which are placed in an orderly arrangement, such as rows and columns.

As described above, it is likely that mixtures of single agents are required in order to achieve a desired cell fate. For example, growth effector molecules that bind to cell surface receptors and regulate the survival, differentiation, proliferation or maturation of these cells include growth factors, extracellular matrix molecules, peptides, hormones and cytokines, of which there are many examples. It can therefore be a tedious task to find the right growth effector or growth effector combinations to place in contact with the cell to achieve a desired cell fate.

The present invention solves a need in the art by providing a high throughput, computer-implemented method to identify optimal agents for a given cell type.

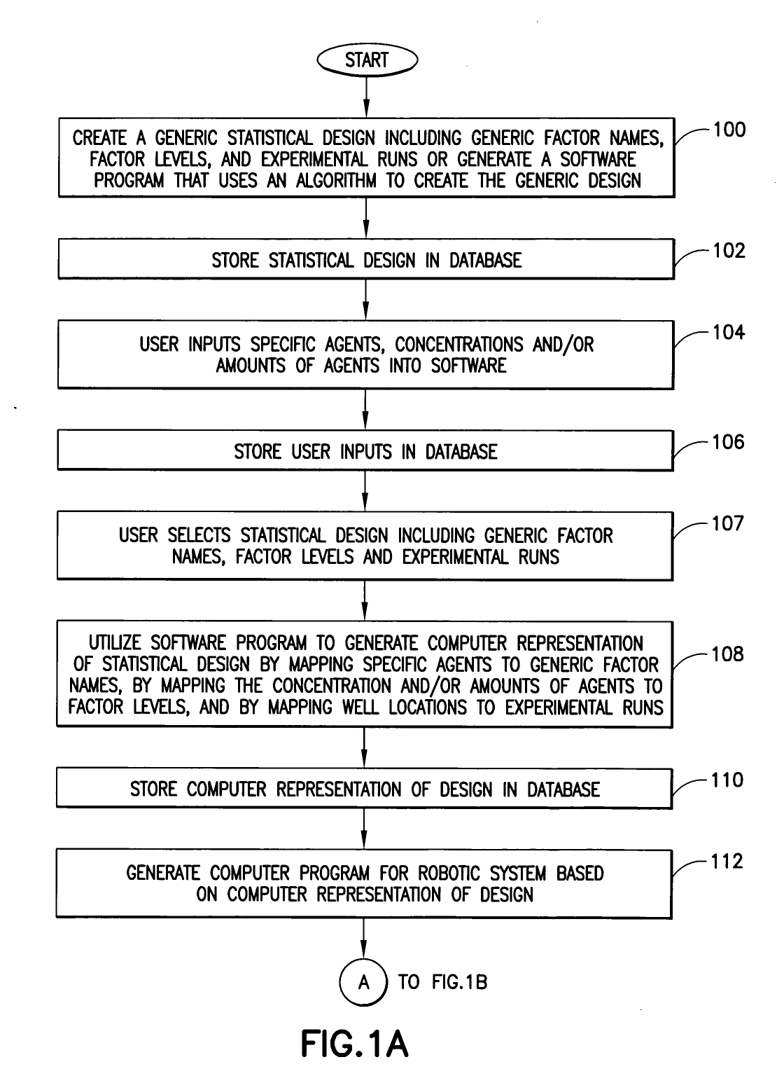

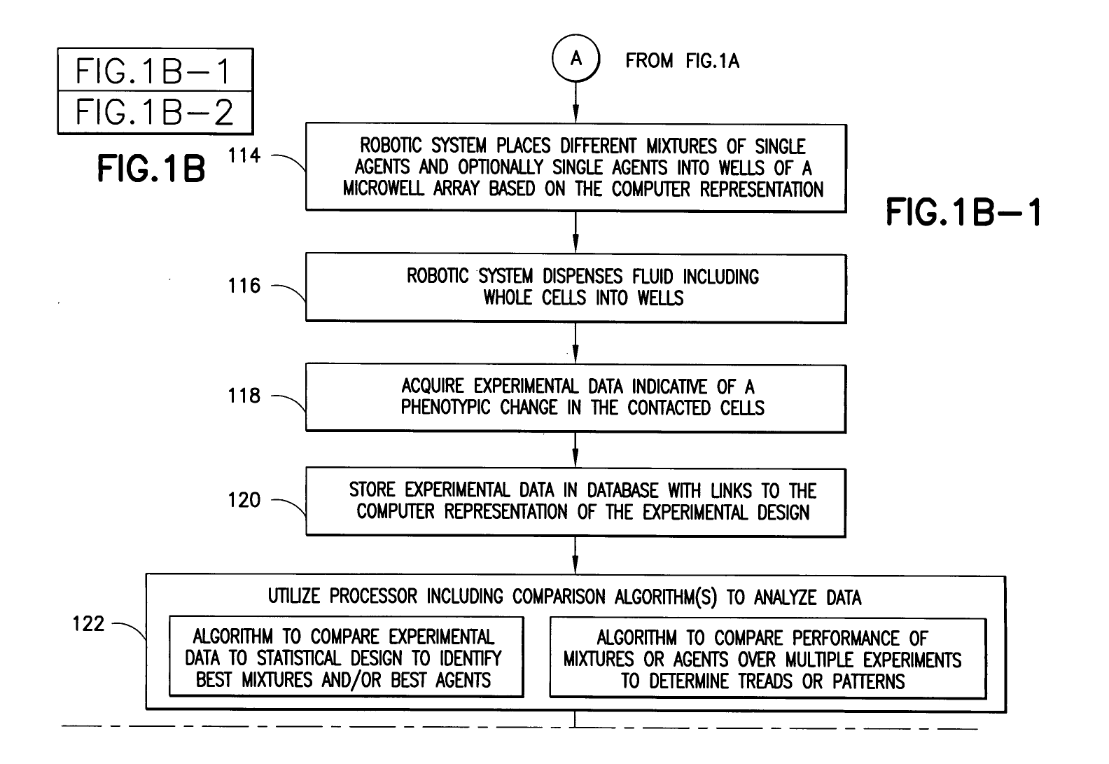

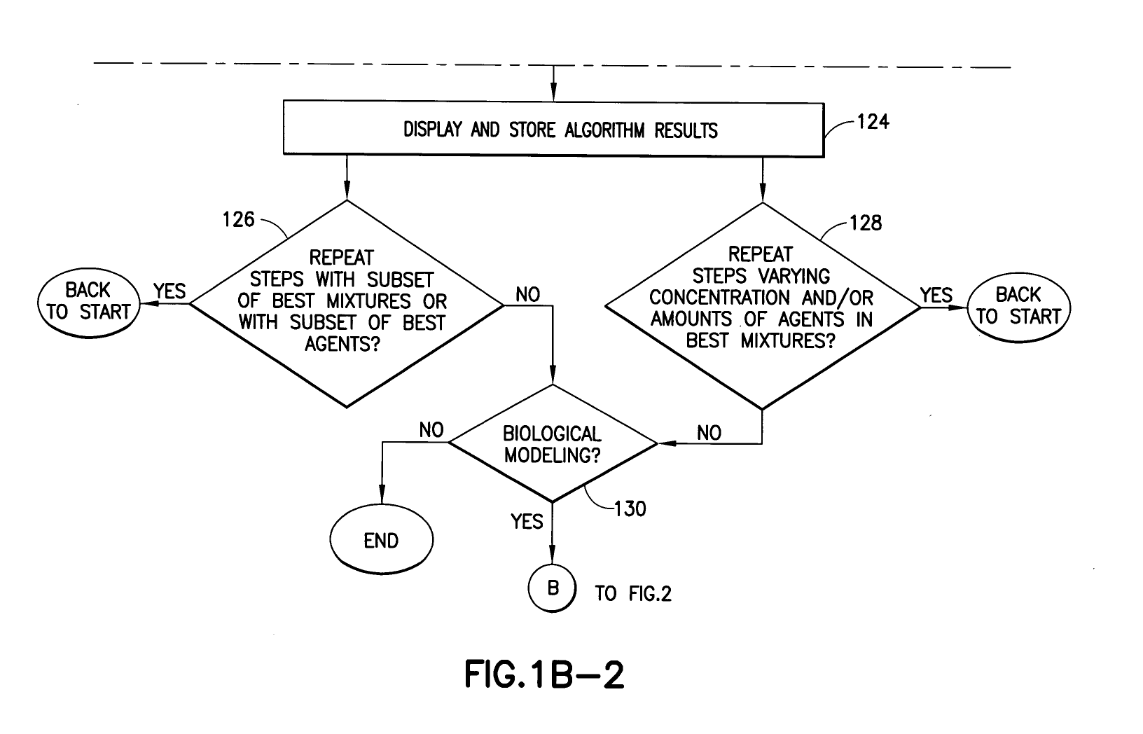

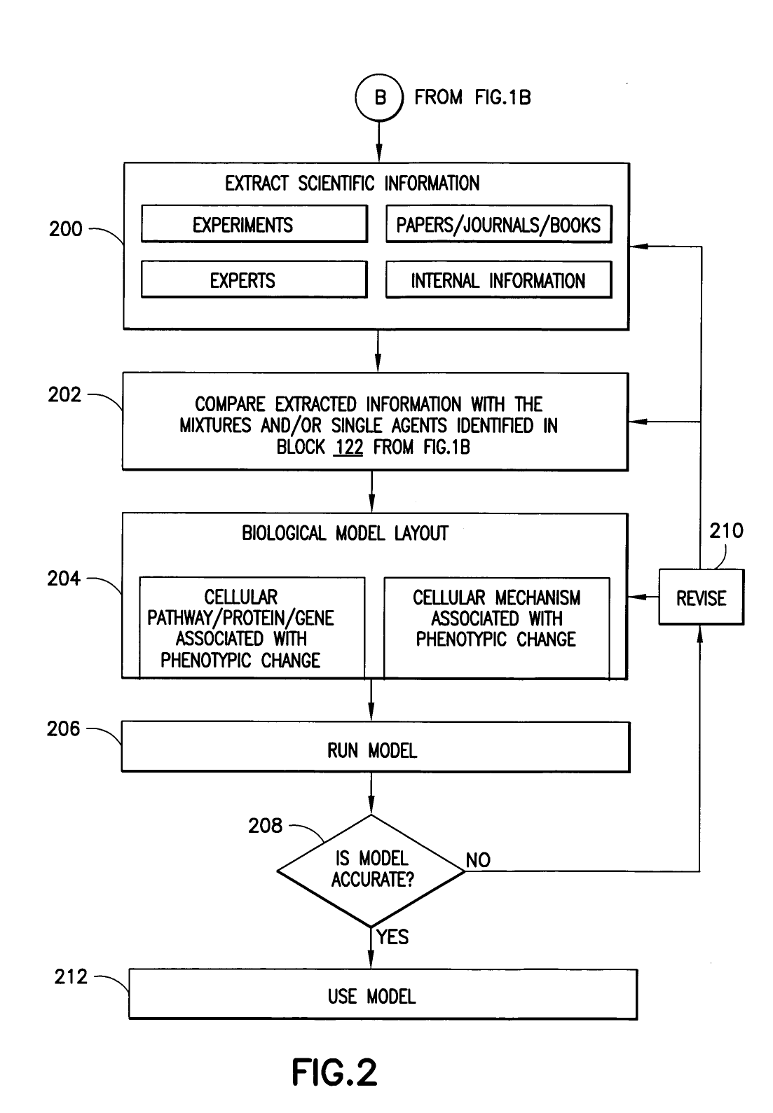

FIGS. 1A and 1B are a schematic flow diagram of a preferred process according to the present invention by which unique mixtures and/or single agents can be identified that are capable of eliciting a change in the phenotype of a cell. In this embodiment, the format used is that of a microwell array. In general, such arrays are well suited to automation, since automatic pipetters and plate readers are readily available. In the first step, a user at block 100 either creates an experimental design using commercially available software such as JMP™ (SAS Institute, Cary, N.C.) or generates a statistical design based on an algorithm that is already included in the software of the system of the present invention. The design at block 100 includes generic factor names, factor levels, experimental runs, and a mapping of experimental runs to a generic microwell array. The statistical design is stored in a database at block 102. The user then inputs at block 104 the specific agents, as well as their concentrations and/or amounts into software. The user inputs are stored in a database at block 106. At block 107, the user can select a specific statistical design. Subsequently, at block 108 a software program is utilized in order to generate a computer representation of the specific statistical design. The computer representation of the design can be a spreadsheet which can translate into a 96-well layout, for example. In particular, the software program used to generate the computer representation maps the names of the specific agents to the generic factor names in the design, maps the concentration and/or amounts of the agents to the factor levels in the design, creates experimental runs based on the specific agents and their concentrations and/or amounts, and maps the well locations to the experimental runs on a specific microwell array.

The computer representation is then stored in a database at block 110. At block 112, a computer program is desirably generated for a robotic system based on the computer representation of the design. At block 114 (FIG. 1B), the robotic system dispenses the agents into the wells according to the computer representation of the design so as to generate different mixtures in select ones of the wells in the microwell array. Optimally, the robotic system can dispense single agents into others of the wells in the microwell array. In addition to reagent addition, withdrawal and wash steps can be performed by the robotic system. Alternatively, some or all of these steps can be performed manually. The agents can be tethered covalently to the well or other culture surface via a biocompatible polymer such as algenic acid or hyaluronic acid or may be present in solution. Once the agents have been placed into the wells correctly, the robotic system at block 116 dispenses fluid including whole cells into the wells of the microwell array. At block 118 experimental data is acquired which would be indicative of a change in the phenotype of a cell. The acquired data is stored in a database at block 120 so that the experimental data is linked to the computer representation of the design. Then, at block 122, a processor is utilized which includes an algorithm to compare the stored experimental data to the stored statistical design to identify the best mixtures and/or best agents, which elicited the desired biological response (i.e., elicited a phenotypic change in the cells). Optionally, another algorithm can be used to compare the performance of mixtures of agents or single agents over multiple experiments to determine trends or patterns. In either case, the results of the algorithm comparisons can be stored in a database and displayed to a user at block 124, and can be periodically updated. In a desired embodiment, the databases shown in FIGS. 1A and 1B are a single integrated or federated database. At block 126, if desired, the steps of the process of the present invention can be repeated with a subset of the best mixtures or a subset of the best agents. Moreover, if desired, the steps can be repeated with a combined subset of best agents and a subset of agents from the best mixtures (not shown). Furthermore, at block 128 the steps of the process of the present invention can be repeated, varying the concentration and/or amounts of the agents in the best mixtures. It is a further aspect of the present invention that information acquired from the algorithm comparisons at block 122 can optimally be used to create or revise a biological model (block 130).

Referring now to FIG. 2, a flow chart is presented of an exemplary process according to the present invention by which a biological model can be created or an existing model can be revised using the information from blocks 122 and 124 of FIG. 1B. At block 200 scientific information is collected from a variety of sources, such as papers, journals, books, experts, experiments, internal information, etc. Scientific information can include, but is not limited to, gene expression data, protein expression data, cellular phenotype data, signal transduction data, data on cellular pathways, and combinations thereof. Such scientific information can be stored in one or more databases. The information can be computer-extracted, such as via the internet. The extracted information is compared at block 202 with the agent mixtures and/or single agents identified in block 122 from FIG. 1B. Based on this comparison, a biological model can be developed at block 204 or revised. The biological model can define the biological systems involved in the phenotypic change, and any relevant communication mechanisms between biological systems. For example, at block 204, a specific cellular pathway, protein or gene associated with the phenotypic change in the cell may be identified. In one embodiment, the processor described in FIG. 1B further includes a first application program for calculating the likelihood that a cellular pathway, protein, or gene is involved in changes in cellular phenotype associated with an identified mixture of single agents. The cellular pathway, protein, or gene is determined using the extracted scientific information. In FIG. 2, the biological model can be a computer-executable model, which is run at block 206, checked for accuracy at block 208 and revised at block 210, if necessary. Once the model is determined to be accurate, it can be used (block 212). An example of a computer-executable model of a biological system is described in U.S. Pat. No. 5,808,918, the entire contents of which are incorporated herein by reference. Desirably, the model would be able to support computation, updating, comparison and visualization.

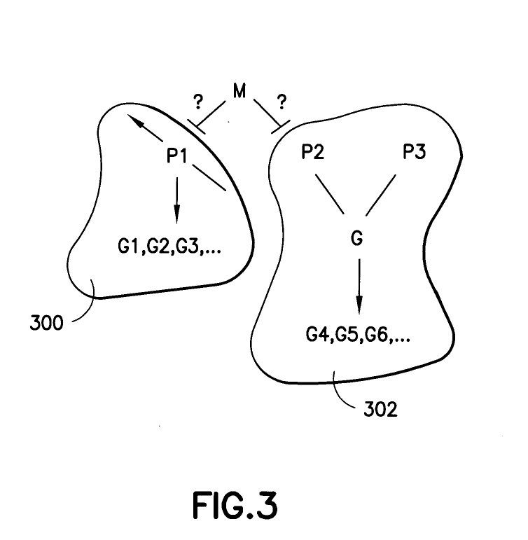

FIG. 3 illustrates exemplary cellular pathways hypothesized for the action of an agent mixture (M) on a biological system. Agent mixture (M) acts on a cell by interacting with hypothetical biological pathways 300 and 302. The arcs between mixture (M) and these pathways represent possible action of mixture (M) on these pathways. The entire action of mixture (M) on the cells assumed to be expressible as a combination of mixture (M)'s actions on one or more of these two pathways. As used herein, a cellular pathway is generally understood to be a collection of cellular constituents related in that each cellular constituent of the collection is influenced according to some biological mechanism by one or more other cellular constituents in the collection. The cellular constituents making up a particular cellular pathway can be drawn from any aspect of the biological state of a cell, for example, from the transcriptional state, or the translational state, or the activity state, or mixed aspects of a biological state. Cellular constituents of a cellular pathway can include mRNA levels, protein abundances, protein activities, degree of protein or nucleic acid modification (e.g., phosphorylation or methylation), combinations of these types of cellular constituents, etc. Each cellular constituent is influenced by at least one other cellular constituent in the collection by some biological mechanism. The influence, whether direct or indirect, of one cellular constituent on another is presented as an arc between the two cellular constituents, and the entire pathway is presented as a network of arcs linking the cellular constituents to the pathway. In FIG. 3, biological pathway 300 includes protein P1 (for example, either the abundance or activity of P1) and genes G1, G2, and G3 (for example, their transcribed mRNA levels). Biological pathway 300 further includes the influence, whether direct or indirect, of protein P1 on these three genes, represented as the arc leading from P1 to these three genes. This influence might arise, for example, because protein P1 can bind to promoters of these genes and increase the abundance of their transcripts. Also shown in FIG. 3 is cellular pathway 302. In this pathway, proteins P2 and P3 both (directly) affect gene G. In turn, gene G (perhaps indirectly) affects genes G4, G5 and G6.

In order to ascertain certain pathways, proteins, or genes of particular interest, aspects of the biological state of the cell, for example, the transcriptional state, the translational state, or the activity state, can be measured in the presence of a mixture of single agents identified as eliciting a phenotypic change in the cell (box 122 of FIG. 1B). In one embodiment, cellular pathways or mechanisms can be identified by identifying genes and/or proteins expressed by the cells in the presence of the identified mixture of single agents. In another embodiment, cellular pathways or mechanisms can be identified by identifying receptors on the cells which are activated in the presence of the identified mixture of single agents.

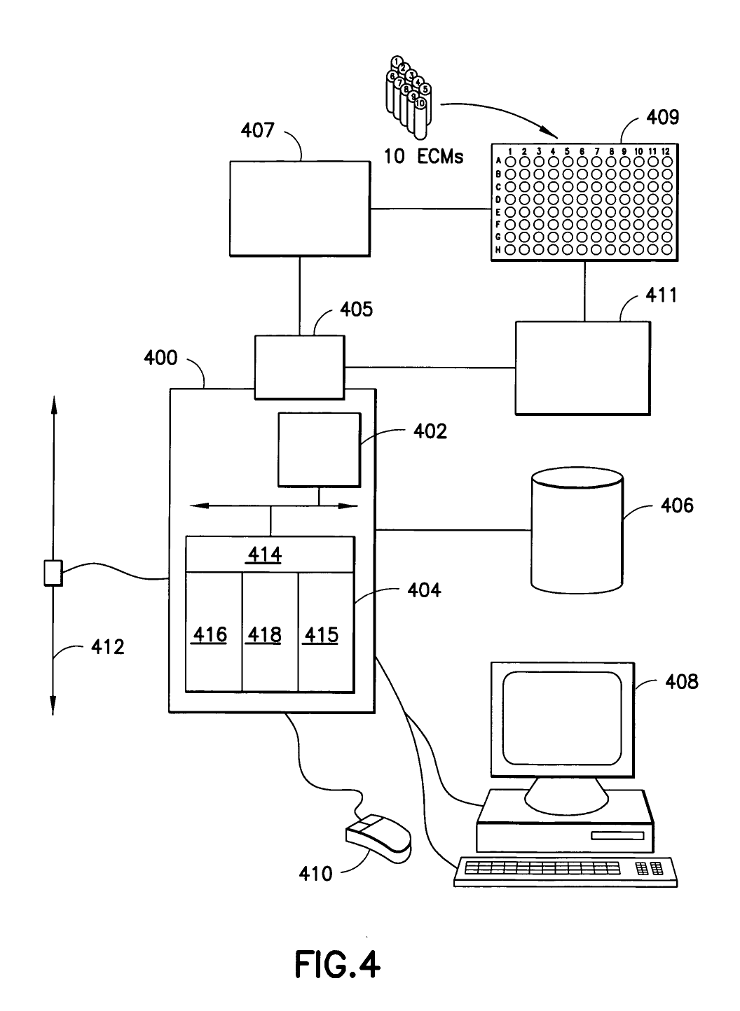

FIG. 4 illustrates an exemplary computer system suitable for implementation of the methods of the present invention. Computer system 400 is shown as including internal components and being linked to external components in the embodiment shown. The internal components include processor 402 interconnected with main memory 404. In one example, computer system 400 can be an Intel Pentium®-based processor of 200 Mhz or greater clock rate and with 32 Mb or more of main memory. External components can include one or more hard disks 406, which are typically packaged together with the processor and memory. The external components further include interface board 405, microwell plate reader 407 and microwell array 409, which together allow experimental data to be communicated to computer system 400. The external components further include robotic system 411, which places experimental factors, such as the ten extracellular matrix proteins (ECMs) shown in FIG. 4 into the receptacles of microwell array 409 in accordance with a statistical design selected by a user. Other external components can include user interface device 408, which can be a monitor and keyboard, together with pointing device 410, which can be a “mouse”, or other graphic input devices (not illustrated). Typically, computer system 400 is also linked to network link 412, which can be part of an Ethernet link to other local computer systems, remote computer systems, or the Internet. This network link 412 allows computer system 400 to share data and processing tasks with other computer systems.

Loaded into the memory 404 are several software components which are both standard in the art and particular to the present invention. These software components collectively cause the computer 400 system to function according to the methods of the present invention. The software components are typically stored on hard disks 406. Software component 414 represents the operating system, which is responsible for managing computer system 400 and its network interconnections. An example of a suitable operating system is Windows 98, or Windows NT. Software component 415 is for analyzing the image from the microwell plate reader 407. Software component 416 represents common languages and functions conveniently present on system 400 to assist programs implementing the methods which are specific to the present invention. Languages that can be used to program the analytical methods of the present invention include Java® in the preferred configuration, but may also include C, C⁺⁺, Fortran, Visual Basic or other computer languages. Most preferably, the methods of the present invention are programmed in mathematical software packages which allows symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), S-Plus from Mathsoft (Seattle Wash.), MathCAD from Mathsoft (Cambridge, Mass.) or “R” from the R Foundation (www.r-project.org). Accordingly, software component 418 represents the methods of this invention as programmed in a procedural language or symbolic package.

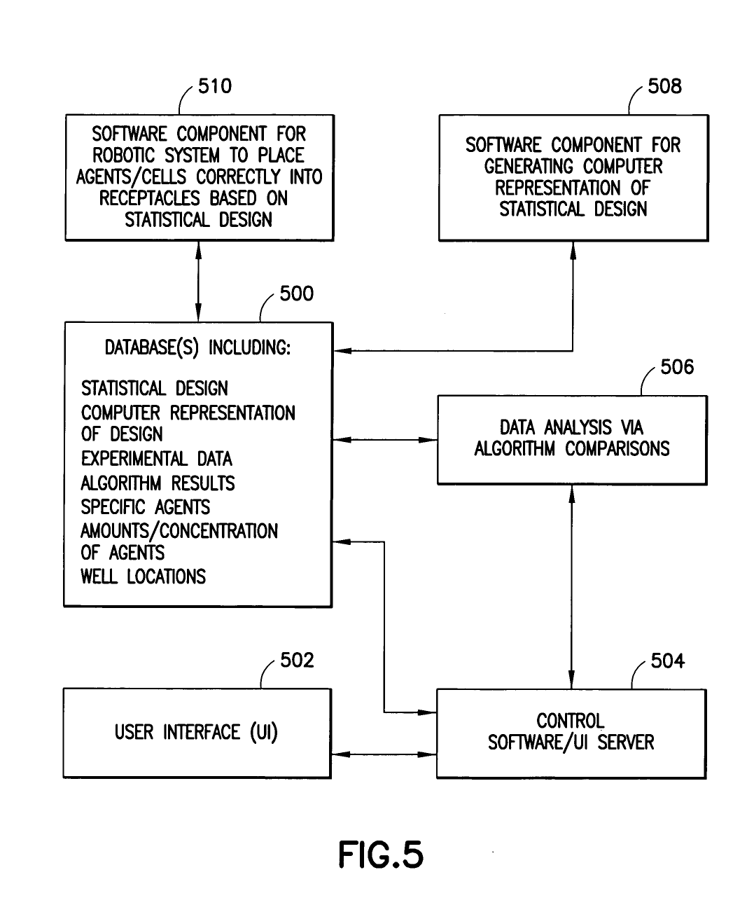

In preferred embodiments, software component 418 actually includes several software components which interact with each other as illustrated in FIG. 5. Software component 500 represents a database, which is preferably a single integrated or federated database containing data necessary for the operation of computer system 400. Such data will preferably include the statistical design, the computer representation of the statistical design, experimental data, algorithm results, the names of specific agents tested, amounts and/or concentrations of the agents tested, and well locations which are to be used in the experiment. Software component 502 represents a user interface (UI), which is preferably a graphical user interface (GUI), which is a graphical way to represent the operating system, such as Windows 2000 or X11. User interface 502 provides a user of the computer system 400 with control and input as to the statistical design, specific agents, their concentrations and/or amounts, and, optionally, experimental data. The user interface may also include a means for loading information, such as experimental data from the hard drive 406, from removable media (e.g., CD-Rom), or from a different computer system communicating with the instant system over a network, such as the Internet. Software component 504 represents the control software, which can be referred to as a UI server, which controls the other software components of the computer system. Software component 506 represents a data reduction and computation component including algorithms which execute the analytic methods of the invention. For example, component 506 can include an algorithm for comparing acquired experimental data to the statistical design to identify the best mixtures and/or best agents. The data can be imported into the software and automatically linked to the statistical design so that the data is filly annotated and ready for statistical analysis. Moreover, component 506 may include an algorithm to compare the performance of mixtures or agents over multiple experiments to determine trends or patterns, which can be stored and periodically updated if desired. In one embodiment, software component 506 includes a linear regression algorithm. This is a method by which coefficients are estimated for each of the specific agents that are used in the statistical design.

Software component 418 also includes a software component 508 for generating a computer representation of the statistical design, as well as a software component 510 for a robotic system to place agents correctly into the wells of array 409 based on the statistical design stored in database 500. For example, a user can select an option via user interface 502 to generate computer files that can be imported into a robotic sample preparation platform, such a Biomek FX, Biomek 2000, Tecan Genesis, or any similar platforms. The computer files can be used to automatically prepare the correct experimental conditions on the microwell array, to culture the cells, and to perform any fluid dispensing, fluid withdrawing or wash steps to carry out assays of phenotype.

It is noted that it is well within the contemplation of the present invention that the method of the present invention could be implemented from a customer location that is remote from the actual laboratory where the experiments are being performed. This could involve a web-based interface or the distribution of a thick-client software application to the customer. The level of interaction between the laboratory and the customer could vary. For example, the customer could have complete control of the process. Alternatively, the customer could receive only periodic reports from the laboratory as to its progress in obtaining optimal mixtures of agents.

In one embodiment of the method of the present invention, mixtures of single agents are covalently immobilized to an agent-immobilizing material on a culture surface, such as the receptacle surface or the surface of a scaffold contained within the receptacle. It is also well within the contemplation of the present invention that mixtures of single agents can be passively adsorbed onto a culture surface. Moreover, some or all of the single agents in the mixture can be in solution.

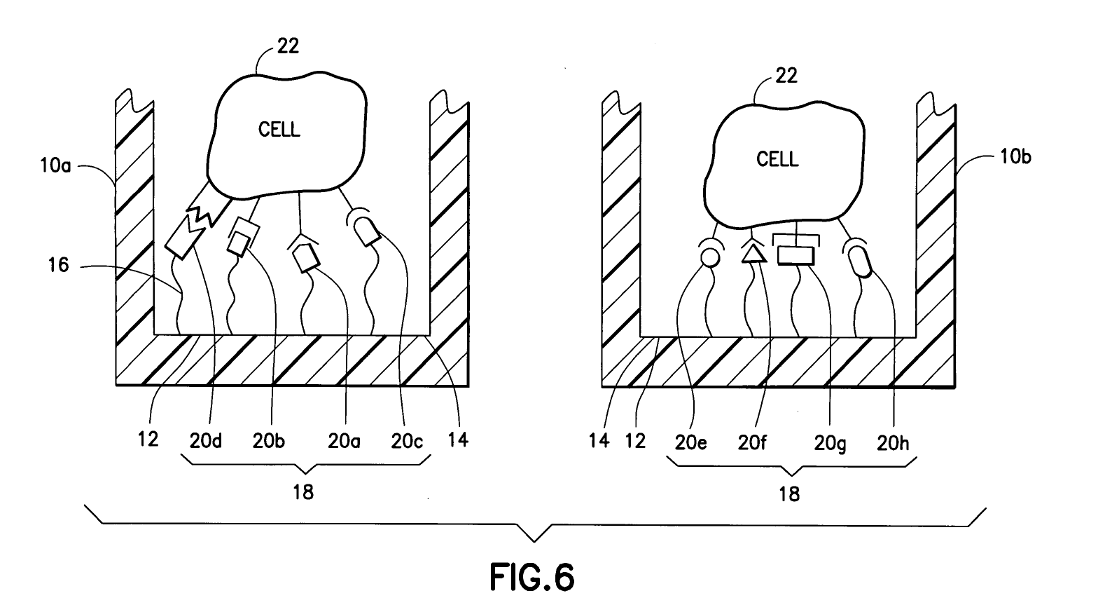

With reference now to FIG. 6, according to the present invention, receptacles 10 are provided in an array (not shown). Receptacles 10 include a surface 12, which can be pretreated. In one embodiment, surface 12 is amino-plasma treated so as to create an amine-rich surface 14 onto which agent-immobilizing material 16 can be attached. As will be described in further detail below, agent-immobilizing material 16 is preferably a biocompatible polymer which has been coupled to aminated surface 14. In the embodiment shown, mixtures 18 of single agents 20, e.g., 20a-d are covalently immobilized to agent-immobilizing material 16. However, it is also well within the contemplation of the present invention that some or all of the agents being tested are in solution, rather than bound to a culture surface. As described above, suitable agents for testing include, but are not limited to, growth factors extracellular matrix molecules, peptides, hormones, and cytokines. Moreover, small molecules, metals, chelators or enzymes can be added as agents to the wells. Different mixtures 18 of single agents 20 are placed into the receptacles 10 according to a statistical design, which will be described in greater detail below. As shown in FIG. 6, the composition of agents 20a-d in receptacle 10a is different from that in a second receptacle 10b, where the composition comprises single agents 20e-h. It is noted, however, that more than one receptacle can include the same agent. For example, it is well within the contemplation of the present invention that a given agent may have a positive effect on achieving a desired cell fate when surrounded by a certain combination of other agents, and that this same agent may have a neutral effect or no effect on achieving a desired cell fate when surrounded by a different combination of agents, Therefore, it would be of benefit to provide an agent in different compositions with other agents to assess these effects. Referring again to FIG. 6, once agents 20 have been placed as different mixtures into the various receptacles 10 according to a statistical design, these mixtures 18 are contacted with whole cells 22. Agents 20 bind to cells 22 and are capable of producing the desired biological response in the contacted cells. A determination as to the effectiveness of a given mixture of agents or of single agents within the mixture at eliciting the desired response in the cell-type is ascertained based on acquired experimental data. Said data can be acquired using methods including, but not limited to, immunocytochemistry analysis, microscopy or functional assays.

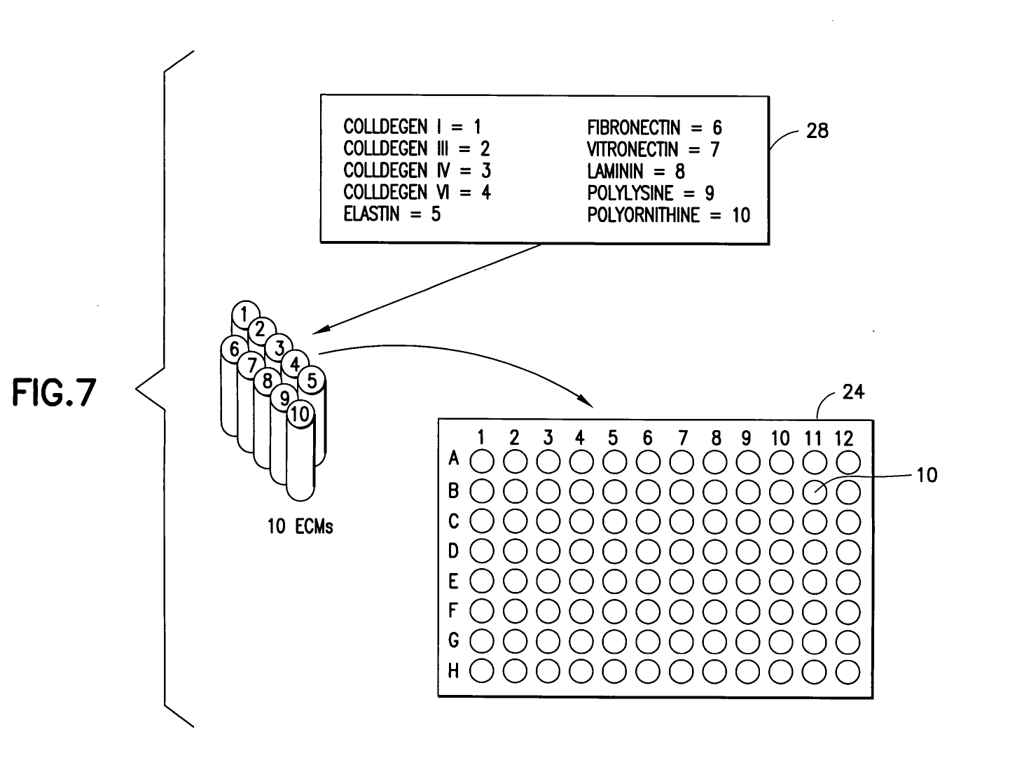

Referring now to FIGS. 7-9, aspects of the statistical design will now be described in further detail. Referring in particular to FIG. 7, receptacles 10 are shown which correspond to a microwell array 24, such as a 96-well plate which is comprised of rows A-H and columns 1-12. As shown in FIG. 7, it is one aspect of the present invention that the identity of single agents 20 or mixtures 18 in FIG. 6 is represented by generic factor names. The factors are the variables in the experiment.

For example, in the embodiment shown in FIG. 7, generic factors 1-10 are representative of the ten single extracellular matrix proteins indicated in box 28. In this example, generic factor 1 is Collagen I, generic factor 2 is Collagen III, etc. Each of these factors can be combined with one or more of the other factors to generate mixtures for the plate layout.

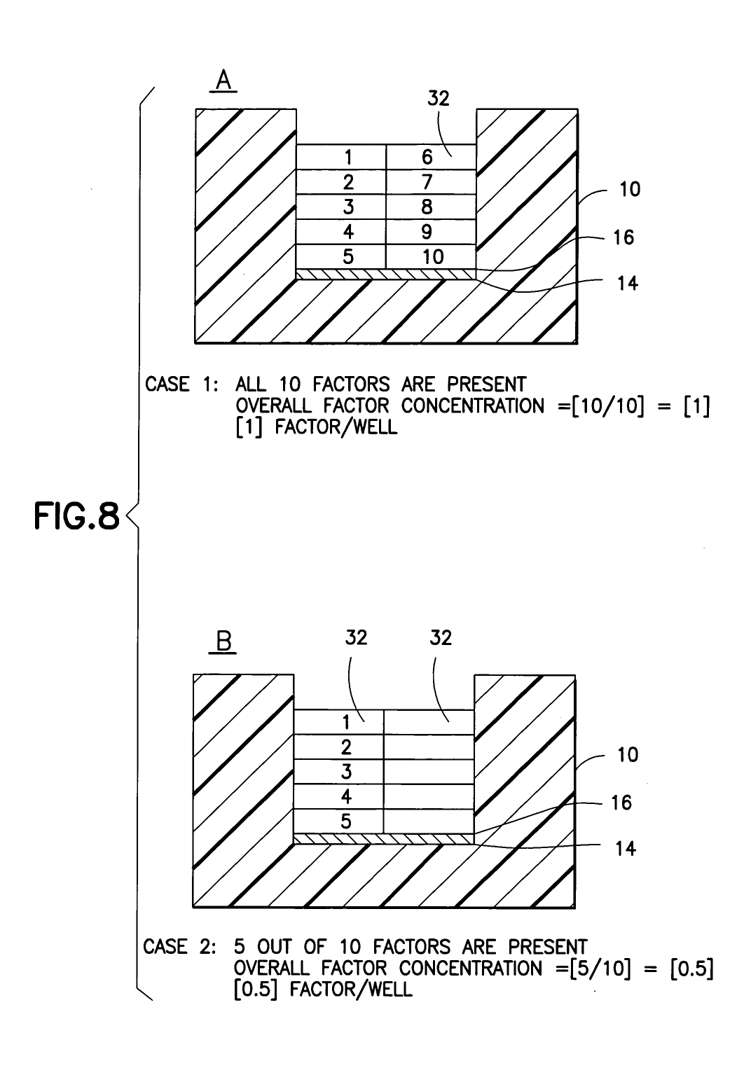

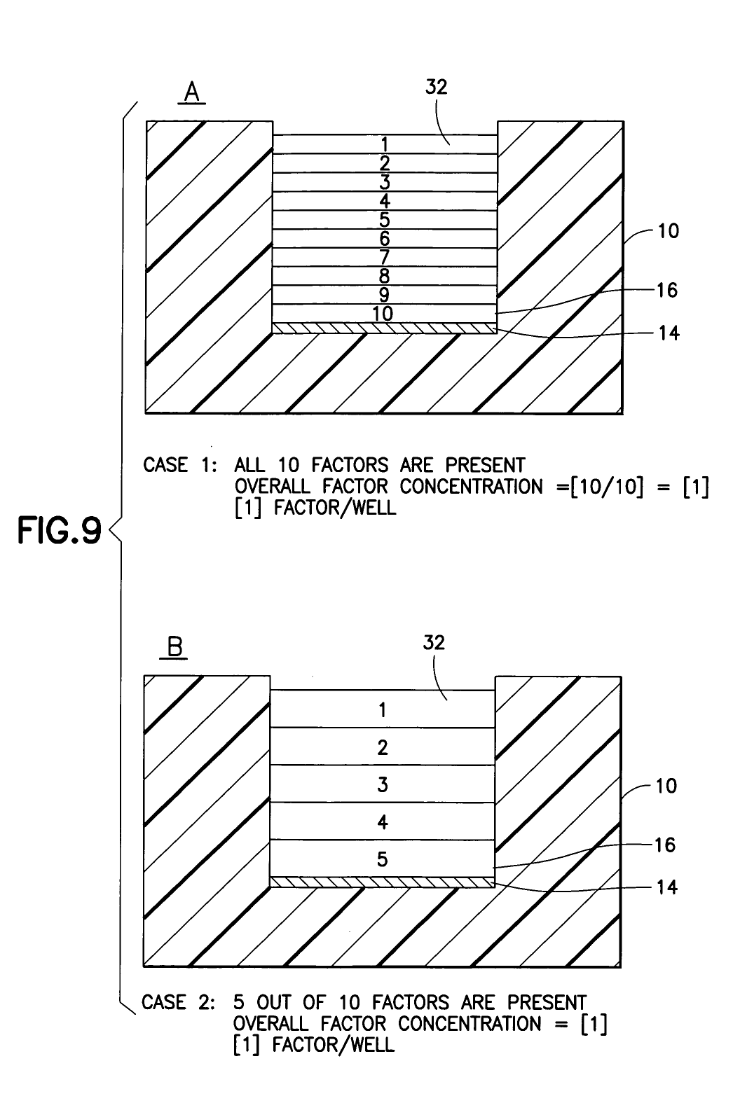

FIGS. 8 and 9 will now be described with reference to the embodiment shown in FIG. 7, wherein each of generic factors 1-10 corresponds to a single agent at a given concentration or amount (i.e. factor level).

As shown schematically in FIG. 8, a scenario is presented in which the total fluid volume within receptacle 10 is divided into ten equal volume compartments 32. Each well of a 96-well plate may contain all ten factors (e.g., single agents) or a subset of these factors. As shown in FIG. 8A, in case 1, all ten factors are present and all ten factors occupy a fluid compartment 32. The overall factor concentration in well 10 shown in FIG. 8A, is [10/10]=[1]. This provides an overall concentration of factor equivalent to [1] per well. FIG. 8B represents a different well on the same 96-well plate, for example. In this situation (case 2), only five out of the ten factors are present. Again, the fluid volume is divided into ten equal compartments 32. In case 2, when a factor is present, the fluid compartment is filled with the factor. However, in case 2, five out of the ten volume compartments are not filled with a factor, but are rather filled with a “place holder”, such as media. In case 2 of FIG. 8B, the overall factor concentration equals [0.5]. Therefore, the overall factor concentration in the wells shown in FIG. 8B is [0.5] factor per well. The overall factor concentration in case 1 is not equivalent to the overall factor concentration in case 2. Therefore, the total concentration of the agents in each receptacle can be different. Moreover, in both case 1 and case 2, the concentration of a single factor is the same between wells. For example, the concentration of factor 1, which can represent a single Collagen I ligand is the same between the well of case 1 and the well of case 2.

With reference now to FIG. 9, another scenario is presented wherein specific consideration is given to the surface chemistry requirements. In particular, in this scenario the overall density of factor is kept constant from well to well and only the factor composition is allowed to change between wells. In other words, the concentration of a factor can be different from well to well, but each well has the same amount of factor immobilized overall. As shown in FIG. 9, the total fluid volume present in a given well is divided up based on the number of factors present. Again, for the sake of simplicity, we can assume that one factor corresponds to one single agent, although the present invention is not limited to this situation. As shown in FIG. 9A, all ten factors are present and the overall factor concentration equals [10/10]=[1] for an overall factor concentration of [1] factor per well. In FIG. 9B, only five out of the ten factors are present, but the fluid volume 32 of each of these five factors is two times that of the volumes 32 of each of the factors shown in FIG. 9A. Consequently, the overall factor concentration shown in FIG. 9B is the same as that shown in FIG. 9A for a total concentration of [1] factor per well. Therefore, in one desired embodiment, the total concentration of the agents in each receptacle is the same. Based on FIG. 9, it can be seen that whereas the overall factor concentration is constant between the well shown in 9A and the well shown in 9B, the concentration of a single factor can be different between these wells. In particular, with reference to factor 1, which may be representative of Collagen I, the concentration of this single agent in FIG. 9B would be twice that shown in FIG. 9A. Therefore, in a further embodiment of the present invention, the concentration of an individual agent differs between the receptacles.

It is noted that each of the scenarios depicted in FIGS. 8 and 9 are feasible and can be used for screening mixtures of single agents.

The present invention provides for methods which use a format, such as a microwell array, to screen a plurality of different mixtures of agents in parallel for their ability to bind to a given cell-type and elicit a desired response in the cell. The method includes placing different mixtures of agents into selective wells of a multi-well plate according to a statistical design. The method optionally includes the step of placing single agents into other of the wells. The method also includes delivering a fluid sample comprising a cell-type to the wells. After an appropriate incubation time between the cells and the samples in the various wells, evidence of an interaction between the cells and the well components can be detected, either directly or indirectly. For example, data can be acquired using functional assays, immunocytochemistry, or microscopy.

Suitable statistical designs for use with the present invention include, but are not limited, to the following: fractional factorial design, D-optimal design, mixture design and Plackett-Burman design. The statistical design can also be a space-filling design based on a coverage criteria, a lattice design, or a latin square design.

As described above, agents can either be bound to a culture surface (e.g., receptacle surface or scaffold surface) or can be in solution. For example, in one embodiment, the culture surface, which may be pre-treated, is coated with an agent-immobilizing material. The agent-immobilizing material is desirably a biocompatible polymer which does not support cell adhesion and which can serve as a flexible link (tether) between the culture surface and the agents. Examples of suitable polymers include synthetic polymers like polyethylene oxide (PEO), polyvinyl alcohol, polyhydroxylethyl methacrylate, polyacrylamide, and natural polymers such as hyaluronic acid and algenic acid.

In desired embodiments, culture surfaces (e.g., well surfaces) are selected from, but not limited to, the following: polystyrenes, polyethylene vinyl acetates, polypropylene, polymethacrylate, polyacrylates, polyethylenes, polyethylene oxide, glass, polysilicates, polycarbonates, polytetrafluoroethylene, fluorocarbons, and nylon. It is also well within the contemplation of the present invention that the culture substrates may wholly or partially include biodegradable materials such as polyanhydrides, polyglycolic acid, polyhydroxy acids such as polylactic acid, polyglycolic acid and polylactic acid-glycolic acid copolymers, polyorthoesters, polyhydroxybutyrate, polyphosphazenes, polypropyl fumurate, and biodegradable polyurethanes.

The culture surfaces can be pre-treated. For example, cell culture surfaces bearing primary amines can be prepared by plasma discharge treatment of polymers in an ammonia environment. In one embodiment, an agent-immobilizing material can be covalently attached to these aminated surfaces using standard immobilization chemistries, as described in copending, commonly owned U.S. application Ser. No. 10/259,797, filed Sep. 30, 2002, the entire contents of which are incorporated herein by reference. Two processes used commercially to create tissue culture treated polystyrene are atmospheric plasma treatment (also known as corona discharge) and vacuum plasma treatment, each of which is well known in the art. Plasmas are highly reactive mixtures of gaseous ions and free radicals. An amino-plasma treatment or oxygen/nitrogen plasma treatment can be used to create an amine-rich surface onto which biocompatible polymers such as hyaluronic acid (HA) or algenic acid (AA) may be coupled through carboxyl-groups using carbodiimide bioconjugate chemistries, as described in U.S. application Ser. No. 10/259,797. The resulting surfaces will not allow cells to attach, even in the presence of high, e.g., 10-20% serum protein concentrations present in the cell culture media. An example of pre-treated tissue culture polystyrene products that can be used to covalently link the agent via the agent-immobilizing material are the PRIMARIA™ tissue culture products (Becton Dickinson Labware), which are created using oxygen-nitrogen plasma treatment of polystyrene and which result in the incorporation of oxygen- and nitrogen-containing functional groups, such as amino and amide groups.

Agents, such as extracellular matrix proteins, peptides, etc. can be subsequently covalently coupled to the HA or AA surface described above utilizing the amine groups on the proteins/peptides and either the carboxyl groups on the HA or AA, or aldehyde groups created on the HA or AA by oxidation using sodium periodate, for example.

For example, the terminal sugar of human placental hyaluronic acid can be activated by the periodate procedure described in E. Junowicz and S. Charm, “The Derivatization of Oxidized Polysaccharides for Protein Immobilization and Affinity Chromotography,” Biochimica et. Biophysica Acta, Vol. 428:157-165 (1976), incorporated herein by reference. This procedure entails adding sodium or potassium periodate to a solution of hyaluronic acid, thus activating the terminal sugar which can be chemically cross-linked to a free amino group on an agent, such as the terminal amino group on an extracellular matrix protein. In another preferred embodiment, free carboxyl groups on the biocompatible polymer (for example, HA or AA) may be chemically cross-linked to a free amino group on the agent using carbodiimide as a cross-linker agent. Other standard immobilization chemistries are known by those of skill in the art and can be used to join the culture surfaces to the biocompatible polymers and to join the biocompatible polymers to the agents. For example, see “Protein Imnmobilization: Fundamentals and Applications” Richard F. Taylor, Ed. (M. Dekker, NY, 1991) or copending U.S. application Ser. No. 10/259,797, filed Sep. 30, 2002.

It is noted that agents can be tethered to aminated tissue culture surfaces via biocompatible polymers, or can be tethered via biocompatible polymers to carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. Again, it is noted that the agents can be present in solution and need not be bound to the culture surface.

As described above, it is an aspect of the present invention that mixtures of agents, which can be bound to a culture surface or can be in solution, are contained within selective ones of the receptacles. Moreover, it is a further aspect of the present invention that other receptacles may contain a single agent. The agents may be combined in any desired proportions. The relative amounts of different agents present in the receptacles can be controlled, for example, by the concentration of the agents in a composition which is to be dispensed into the receptacles. Moreover, in embodiments where the agents are covalently attached via a biocompatible polymer to the receptacle surface, the loading density can be controlled by adjusting the capacity of the biocompatible polymers bound to the culture surface. This can be accomplished by, for example, controlling the number of reactive groups on the polymers that can react with the agents or by controlling the density of the biocompatible polymer molecules on the culture surface. Furthermore, the agents can first be separately linked to the biocompatible polymers (tethers), and then the “loaded” tethers can be mixed in the desired proportions, and attached to the pre-treated substrate.

As described above, the agents can be in solution and/or can be bound to a surface. For example, the agents can be covalently immobilized via biocompatible polymers to a pre-treated tissue culture surface, which is desirably amine-rich. Alternatively, the agents can be immobilized to the receptacle surfaces by passively adsorbing the agents to the surface. It is also well within the contemplation of the present invention that agents can be pre-immobilized onto solid supports, such as beads, which then can be added to the receptacles. A response in a cell-type contacted with the beads in the receptacles could subsequently be detected. Mixtures of beads comprising single agents may be combined to form agent mixtures. Alternatively, mixtures of single agents can be immobilized to the beads.

It is well within the contemplation of the present invention that the agents can be immobilized on or impregnated within a scaffold, which can be placed in the receptacle and then contacted with fluid containing the cells. Suitable scaffolds for use n the present invention and methods for immobilizing agents thereto or therewithin are described in copending, commonly owned U.S. application Ser. No. 10/259,817, filed Sep. 30, 2002, the entire contents of which are incorporated herein by reference.

Receptacles for use in the present invention can take any usual form, but are desirably microwells or tubes. Configurations such as microtitre wells and tubes are particularly useful in the present invention and allow the simultaneous automated assay of a large number of samples to be performed in an efficient and convenient way. Microtitre wells are capable of extensive automation because of automatic pipetters and plate readers. Other solid phases, particularly other plastic solid supports, may also be used.

In one preferred embodiment of the present invention, the receptacles comprise the wells of a 96-well microtitre plate (i.e., microwell array). Automatic pipetting equipment (for reagent addition and washing steps) and color readers already exist for microtitre plates. An example of an automated device for carrying out the present invention can include: a pipetting station and a detection apparatus (e.g., plate reader), the pipetting station being capable of performing sequential operations of adding and removing reagents to the wells at specific time points in a thermostatic environment (i.e., temperature controlled environment).

As described above, agents for use in the present invention are growth effector molecules that bind receptors on the cell surface or are taken up through ion channels or transports and regulate the growth, replication or differentiation of target cells or tissue. In one embodiment, these agents are cell adhesion ligands and/or extrinsic factors. In desired embodiments, the agents can be extracellular matrix proteins, extracellular matrix protein fragments, peptides, growth factors, cytokines, and combinations thereof.

Preferred agents are growth factors, extracellular matrix molecules, cytokines, peptides, hormones, metals, chelators or enzymes. Examples of growth factors include, but are not limited to, vascular endothelial-derived growth factor (VEGF), epidermal growth factor (EGF), platelet-derived growth factor (PDGF), transforming growth factors (TGFα, TGFβ), hepatocyte growth factor, heparin binding factor, insulin-like growth factor I or II, fibroblast growth factor, erythropoietin nerve growth factor, bone morphogenic proteins, muscle morphogenic proteins, and other factors known to those skilled in the art. Other suitable growth factors are described in “Peptide Growth Factors and Their Receptors I” M. B. Spom and A. B. Roberts, Eds. (Springer-Verlag, NY, 1990), for example.

Growth factors can be isolated from tissues using methods known in the art. For example, growth factors can be isolated from tissue or can be produced by recombinant means. For example, EGF can be isolated from the submaxillary glands of mice and Genentech (South San Francisco, Calif.) produces TGF-β recombinantly. Other growth factors are also available from vendors, such as Sigma Chemical Co. (St. Louis, Mo.), R&D Systems (Minneapolis, Minn.), BD Biosciences (San Jose, Calif.), and Invitrogen Corporation (Carlsbad, Calif.), in both natural and recombinant forms.

Examples of suitable extracellular matrix molecules for use in the present invention include vitronectin, tenascin, thrombospondin, fibronectin, laminin, collagens, and proteoglycans. Other extracellular matrix molecules are described in Kleinmari et al., “Use of Extracellular Matrix Components for Cell Culture,” Analytical Biochemistry 166: 1-13 (1987), or known to those skilled in the art.

Additional agents useful in the present invention include cytokines, such as the interleukins and GM-colony stimulating factor, and hormones, such as insulin. These are described in the literature and are commercially available.

Cells for use with the present invention can be any cells that can potentially respond to the agents or that need the agents for growth. For example, cells can be obtained from established cells lines or separated from isolated tissue. Suitable cells include most epithelial and endothelial cell types, for example, parenchymal cells, such as hepatocytes, pancreatic islet cells, fibroblasts, chondrocytes, osteoblasts, exocrine cells, cells of intestinal origin, bile duct cells, parathyroid cells, thyroid cells, cells of the adrenal-hypothalamic-pituitary access, heart muscle cells, kidney epithelial cells, kidney tubular cells, kidney basement membrane cells, nerve cells, blood vessel cells, cells forming bone and cartilage, and smooth and skeletal muscles. Other useful cells can include stem cells which may undergo a change in phenotype in response to a select mixture of agents. Further suitable cells include blood cells, umbilical cord blood-derived cells, umbilical cord blood-derived stem cells, umbilical cord blood-derived progenitor cells, umbilical cord-derived cells, placenta-derived cells, bone marrow derived cells, and cells from amniotic fluid. The cells can be genetically engineered. The cells are cultured with agents in a receptacle, such as the well of a 96-well microtitre plate. These cells can be cultured using any of the numerous well known cell culture techniques, such as those described in Freshney, “Cell Culture, A Manual of Basic Technique” 3^{rd} Edition (Wiley-Liss, NY, 1994). Other cell culture media and techniques are well known to those skilled in the art and can be used in the present invention. The cells can be cultured in the presence of agents which are in solution or which are bound to a standard tissue culture vessel, such as a microtitre plate. It is also well within the contemplation of the present invention that the cells can be cultured in suspension using agents that have been tethered to beads or fibers, preferably on the order of 10 microns in diameter of length. These particles, when added to culture medium, would attach to the cells, thereby stimulating their growth and providing attachment signals.

Statistically designed experiments in accordance with the present invention will now be described.

### EXAMPLES

### Example 1

Coupling of Hyaluronic Acid to an Amine-Rich Tissue Culture Surface

An oxygen/nitrogen plasma is used by Becton Dickinson Labware to create PRIMARIA™ tissue culture products. In particular, oxygen/nitrogen plasma treatment of polystyrene products results in incorporation of oxygen- and nitrogen-containing functional groups, such as amino and amide groups. For this experiment, HA was coupled to the amine-rich surface on PRIMARIA™ multi-well plates through carboxyl groups on HA using carbodiimide bioconjugates chemistries well known in the art, such as those described in “Protein Immobilization: Fundamentals and Applications” Richard S. Taylor, Ed. (M. Dekker, NY, 1991) or as described in copending, commonly owned U.S. application Ser. No. 10/259,797, filed Sep. 30, 2002.

### Example 2

Coupling of ECM Proteins to Hyaluronic Acid

ECM agents were covalently attached to the HA polymer tethered to the culture surface from Example 1. In particular, aldehyde groups were created on HA by oxidation using the periodate procedure described in E. Junowicz and S. Charm, “The Derivatization of Oxidized Polysaccharides for Protein Immobilization and Affinity Chromotography,” Biochimica et. Biophysica Acta, Vol. 428: 157-165 (1976). This procedure entailed adding sodium periodate to a solution of HA, thus activating the terminal sugar. Subsequently, the activated HA was coupled to the amine groups on the ECM proteins using standard immobilization chemistries, such as those described in “Protein Immobilization: Fundamentals and Applications” Richard F. Taylor, Ed. (M. Dekker, NY, 1991) or copending U.S. application Ser. No. 10/259,797, filed Sep. 30, 2002.

### Example 3

Use of a Statistically Designed Experiment (Mixture Design) to Screen 10 Different ECM Proteins Simultaneously

In the present example, the statistical design is a mixture design. This design was used to identify pairs of factors, or single factors that had a positive effect on a cell response, and allows us to look at interactions between two ECMs. In this example, 10 single ECMs, each representing a single “factor” are used to created ECM mixtures for placement into the wells of a 96-well plate as shown in FIG. 7. The ECMs covalently attach to biocompatible polymers on the culture surface (see Examples 1 and 2). It is noted that without a statistical design for the experiment, it would take 2¹⁰ (1024) single experiments, or eleven 96-well plates, to test each of the 10 ECMs together with the others against a given cell-type.

In this example, a group of 10 adhesion ligands was selected and a 96-well array was chosen as the format for this screen. To eliminate border effects due to uneven evaporation, only the inner 60 wells of the 96-well array are to be used for the experiment. Wells in the outer rows and columns of the plate can thus be used for suitable controls.

The following 10 adhesion ligands were selected based on their common use as cell culture reagents, commercial availability and price: Collagen I (CI), Collagen III (CIII), Collagen IV (CIV), Collagen VI (CVI), elastin (ELA), fibronectin (FN), vitronectin (VN), laminin (LAM), polylysine (PL), and polyomithine (PO).

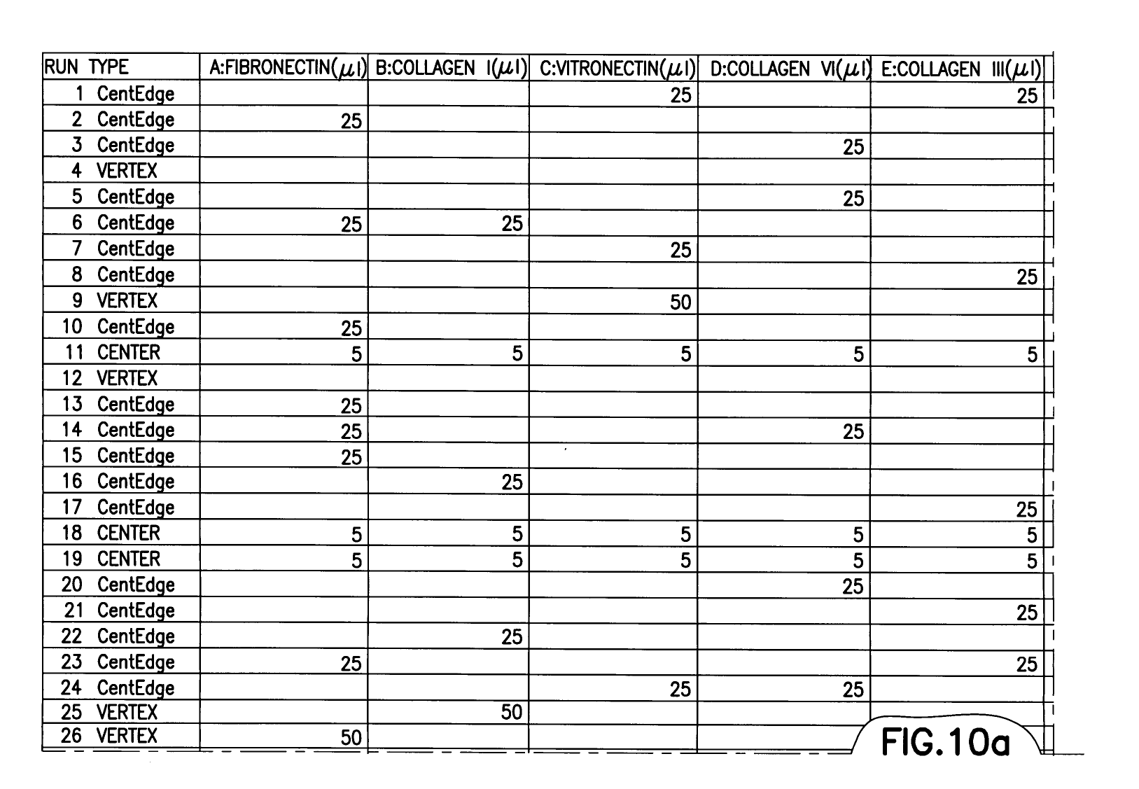

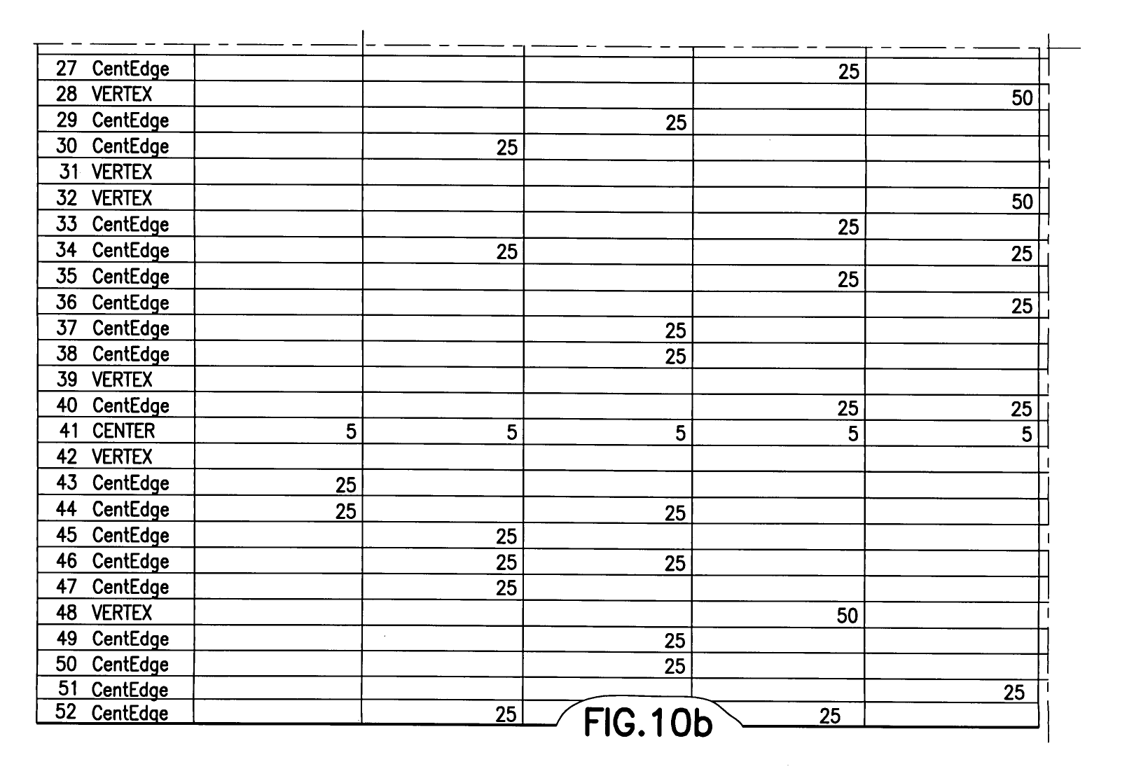

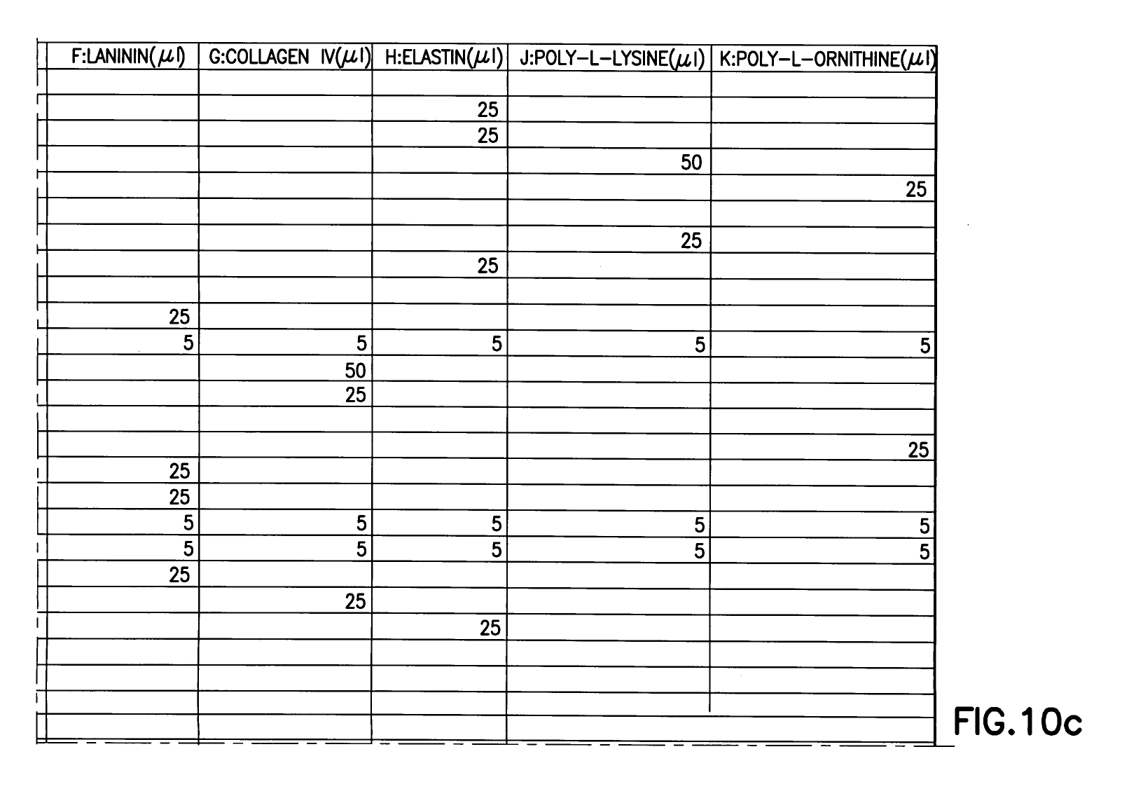

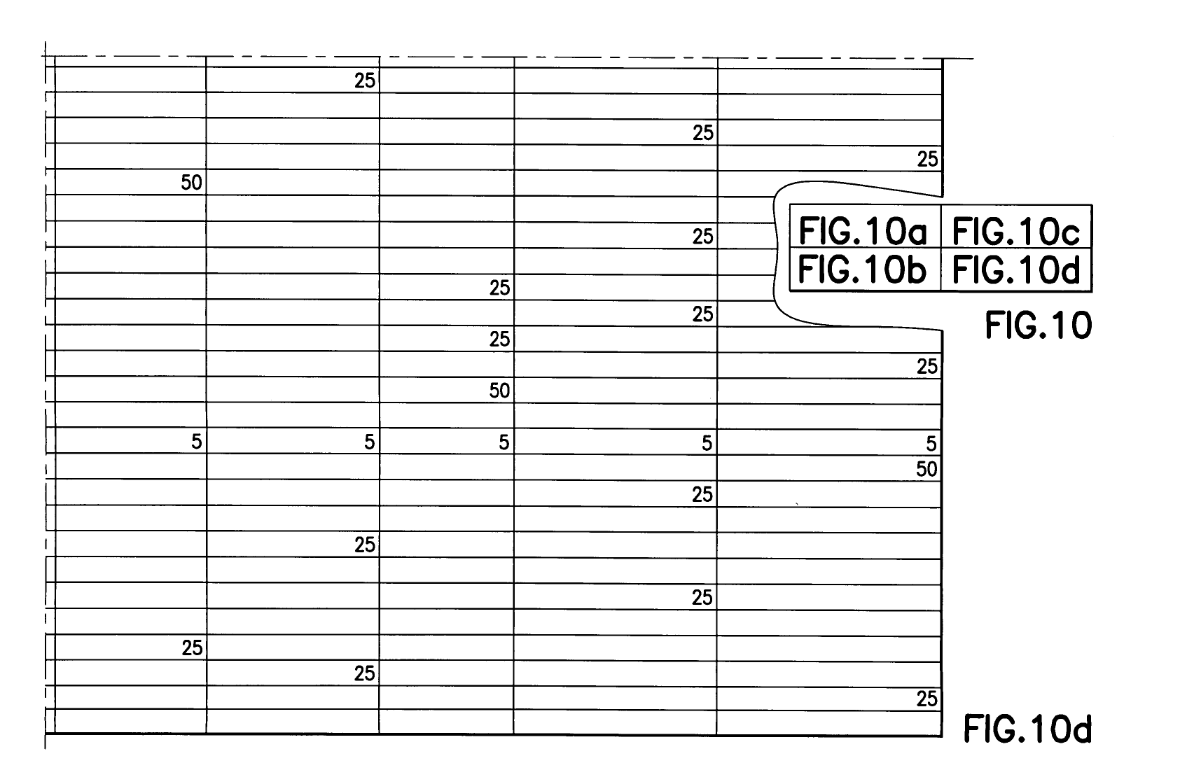

A statistical design was developed with special consideration of the surface chemistry requirements. In particular, in this experiment the scenario shown in FIG. 9 was used, wherein the overall adhesion ligand density was kept constant from well to well and only the adhesion ligand composition was allowed to change. In other words, the concentration of a single adhesion ligand could be different from well to well, but each well has the same amount of adhesion ligand immobilized overall. This scenario is further described above. An example of such design is shown in the spreadsheet in FIG. 10. The spreadsheet serves as a computer representation of the design which is stored in a database. The top row in FIG. 9 lists the 10 factors (A-K) used in this particular screen, and their corresponding identities. In the spreadsheet shown, Factor A represents fibronectin, Factor B represents Collagen I, etc. The first column is a list of the experimental points that translate into a well in the 96-well plate, e.g., 52 wells in this case. The numbers in the spreadsheet are the factor levels. In this example, these levels are the actual volumes (in μL) of factor that are added to a particular well. In this particular design, factors get added to the wells at three volumes, e.g., 5 μL, 25 μL, or 50 μL. The total well volume in this case is 50 μL. Thus, for wells where one factor is added at 50 μL, the final well composition will comprise a single adhesion ligand covalently immobilized on the well surface. Accordingly, if 25 μL of a factor is added to a well, a second factor is added at 25 μL also, and the final well composition will comprise a mixture of two different cell adhesion ligands covalently immobilized on the well surface. When 5 μL of a factor are added, nine other factors are added at 5 μL each, as well, thus resulting in wells that comprise a mixture of all 10 cell adhesion ligands on the well surface. These experimental points containing all 10 adhesion ligands are called “mid points” and are an integral part of the statistical design in this example.

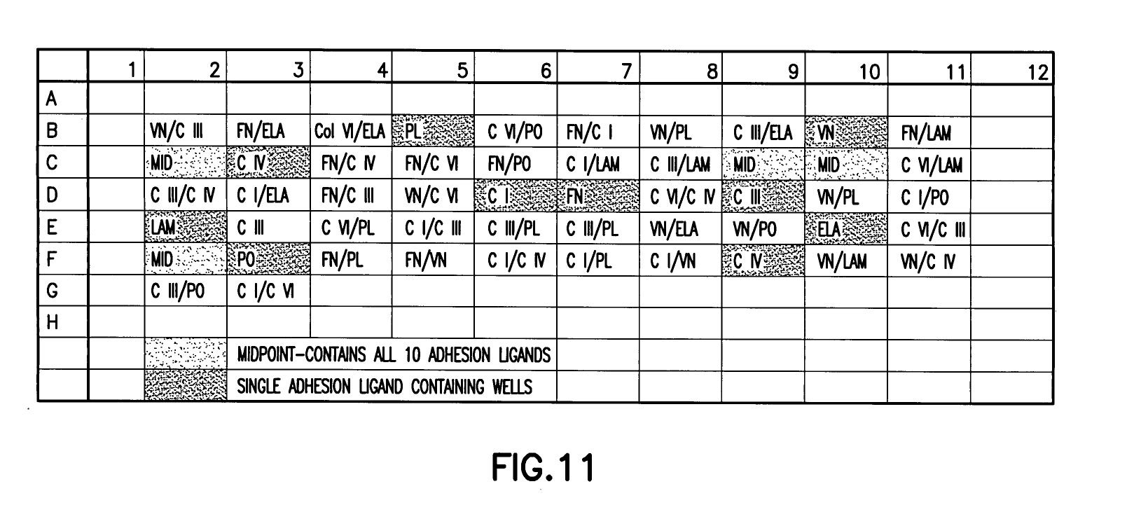

With reference now to FIG. 11, a 96-well plate layout is shown, which was translated from the particular statistical design shown in FIG. 10. In particular, the 96-well plate includes the well compositions indicated in FIG. 10, e.g., cell adhesion ligand combinations immobilized at the bottom of each well. In particular, the experimental runs in FIG. 10 correspond to rows/columns in FIG. 11, as follows: runs 1-10 in the design in FIG. 10 represent row B, columns 2-11, respectively on the array in FIG. 11; runs 11-20 represent row C, columns 2-11; runs 21-30 represent row D, columns 2-11; runs 31-40 represent row E, columns 2-11; runs 41-50 represent row F, columns 2-11; and runs 51 and 52 represent row G, columns 2 and 3, respectively. As shown by the statistical design in FIG. 10 and the corresponding 96-well plate layout in FIG. 11 it is an embodiment of the present invention that, in addition to mixtures of agents, single agents can be placed in the receptacles.

### Example 4

ECM Screen Specific to MC3T3-E1 Osteoblast Cells

MC3T3-E1 cells, originated from Dr. L. D. Quarles, Duke University, and were kindly provided by Dr. Gale Lester, University of North Carolina at Chapel Hill. These cells were grown using standard cell culture techniques. MC3T3-E1 is a well-characterized and rapidly growing osteoblast cell line that was chosen because it attaches aggressively to most commonly used tissue culture surfaces.

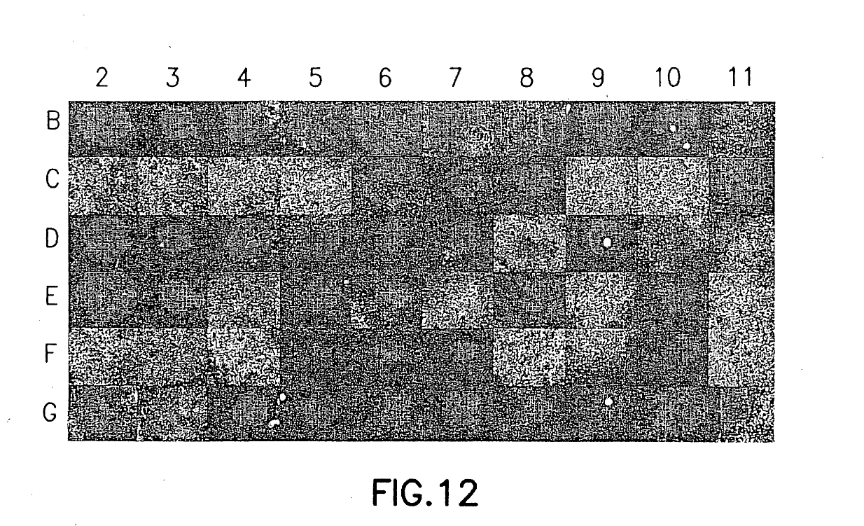

Cells were removed from cell culture flasks using trypsin-EDTA according to methods well known in the art. Cells were enumerated, spun down and resuspended in media containing no serum or, alternatively, in media containing 10% fetal calf serum. Cells were plated into the wells of a 96-well microarray according to the layout shown in FIG. 11 and described in Example 3 above. The seeding density was about 10,000 cells per well. Cells were incubated on the plates overnight at 37° C. The following day, media and any cells not adhering to the immobilized agents on the well surfaces were removed. Any adhered cells were fixed by exposure to forrnalin for at least 15 minutes. Propidium iodite was used to fluorescently label the nuclei of said fixed adhered cells. A fluorescent microscope (Discovery-1, Universal Imaging Corporation, a subsidiary of Molecular Devices, Downingtown, Pa.) was used to acquire images of the fluorescently labeled cells attached to the wells in the ECM screening plate. An example of an image acquired from a 96-well plate is shown in FIG. 12. In particular, the layout is the same as that shown in FIG. 11, except that row G, column 4-11 are used as control wells. In FIG. 12, MC3T3-E1 cells in 10% fetal calf serum-containing media were placed into wells containing mixtures of agents that had been tethered to a hyaluronic acid surface, with the exception that wells G4-G9 contained a hyaluronic acid surface only and wells G10 and G11 comprised tissue culture grade polystyrene only. As expected, the hyaluronic acid surface only in wells G4-G9 prevented cell adhesion. Cell adhesion to the polystyrene surfaces in wells G10 and G11 was, in this example, surprisingly low. In contrast, some wells containing cell adhesion ligands showed strong cell adhesion, as can be seen by the large number of white spots, each of which represents the nucleus of an adhered cell.

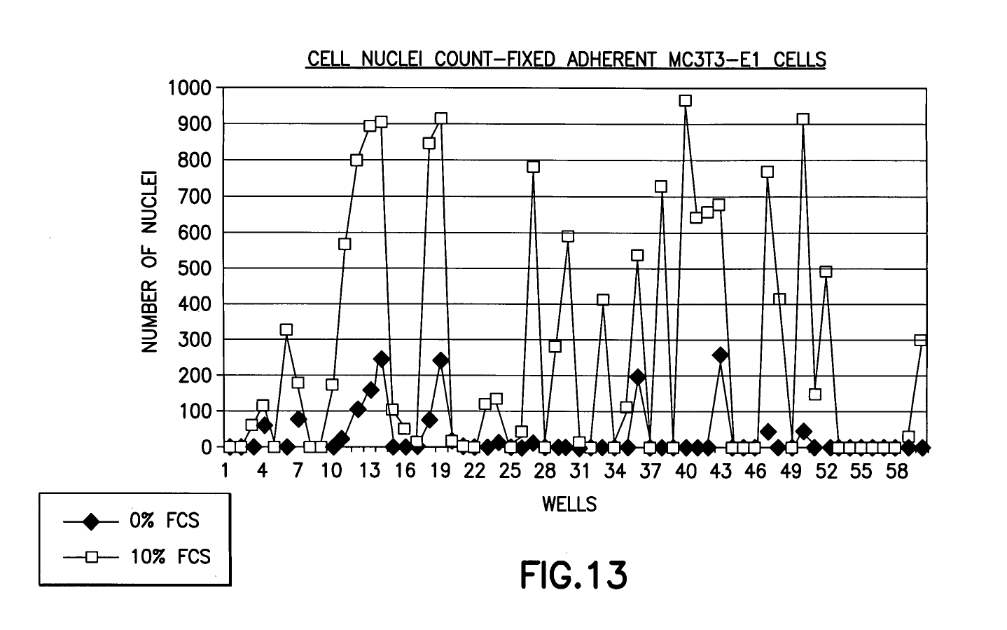

An image analysis software package (Meta Morph, Universal Imaging Corporation, a subsidiary of Molecular Devices, Downingtown, Pa.) was used to enumerate the fluorescently labeled cell nuclei in FIG. 12 and the nuclei count results for both cells in media containing no fetal calf serum and media containing 10% fetal calf serum are shown in FIG. 13. In FIG. 13, wells 1-10 correspond to row B, columns 2-11 in FIG. 9; wells 11-20 in FIG. 12 correspond to row C, columns 2-11 in FIG. 12, etc.

In FIG. 13, in the presence of 10% fetal calf serum, cell adhesion was observed for a number of wells. In the absence of serum, cell adhesion was reduced, but cell adhesion was still observed in a number of wells. In both cases, cell adhesion in some wells containing cell adhesion ligands according to the statistically designed experiment exceeded that of cells cultured on plain tissue-culture grade polystyrene (wells 59 and 60 in FIG. 12). The results obtained enabled the identification of a number of surfaces that support MC3T3-E1 adhesion better than tissue culture grade polystyrene, the most commonly used cell culture support.

In order to optimize the surfaces, one can follow two leads, e.g., the “best well” composition or the “best factors”. The determination of “best factors” is made following rigorous statistical analysis of the experimental results.

In the “best well” approach, the well with the best experimental outcome is chosen for further optimization. In the example shown in FIG. 13, one would choose well 40 (or well E11) which had the highest number of cell nuclei. This well contained a mixture of Collagen-type VI and Collagen-type III according to the plate layout shown in FIG. 11. The concentration of Collagen-type VI and Collagen-type III that was chosen for the immobilization step in the ECM screening plate preparation was based on initial concentration-dependent studies with the MC3T3-E1 cells using the model ECM, fibronectin. It is noted that a concentration which is optimal for one cell-type under investigation may not be optimal for another cell-type. Moreover, the concentration of a particular ECM which is optimal for a given cell-type may not be the optimal concentration for another ECM, even when the same cell type is used. Similarly, the composition of a mixture in the “hit well” may not be optimal. For example, the surface of well E11, which was the “best well” comprised a 50/50 mixture of Collagen-type VI and Collagen-type III. Follow-up experiments may be performed to optimize the concentration of both ligands chosen for the immobilization step, as well as the composition of the mixture (a 50/50 mixture may not be the optimal composition) bound to the surface of a “hit” well for a given cell-type.

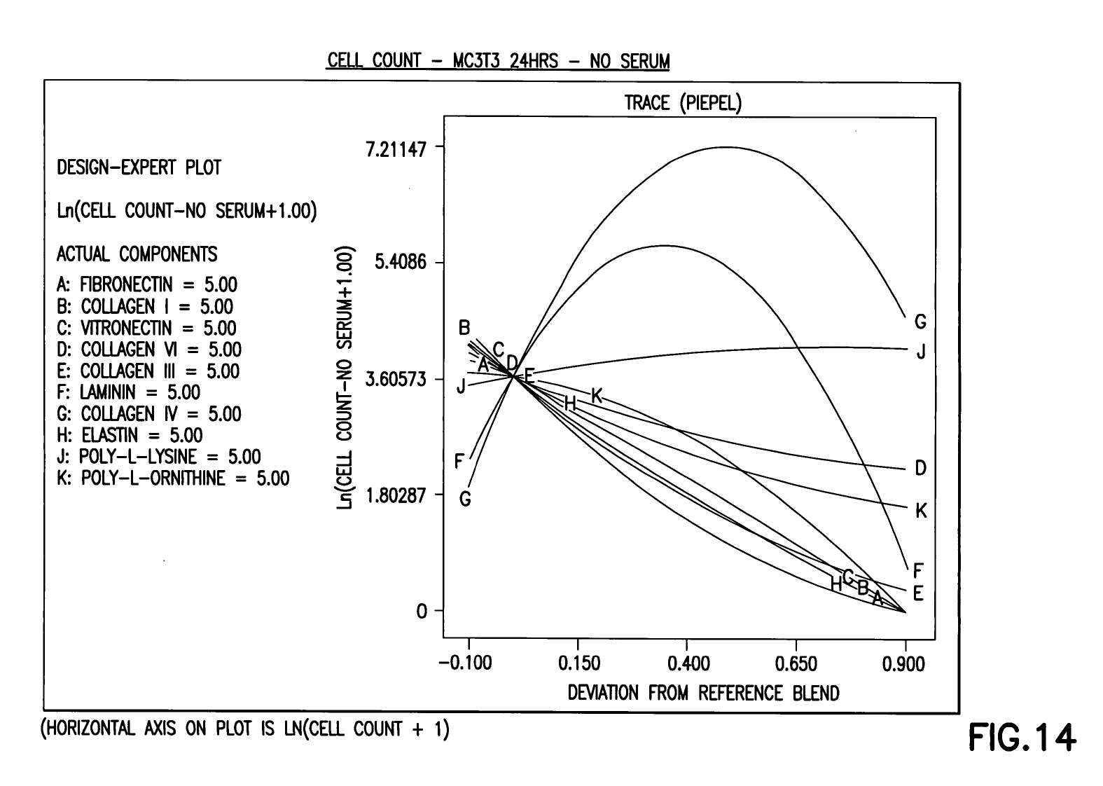

In the “best factors” approach, the experimental results are analyzed using statistical models. For the above-described example, a mixture-model analysis of the MC3T3-E1 data shows that Collagen IV, laminin, and poly-L-lysine (marginal effect) appear to increase the cell count when present at significant quantities with no serum as shown in FIG. 14. The points at which all the lines intersect correspond to mid-points, where all 10 ECMs were present at 5 μL each. This graph provides an indication as to how the cell count changes, depending on how far the well composition deviates from this reference “mid-point” blend. As can be seen, as the amount of Collagen IV or laminin increases, the cell counts increase.

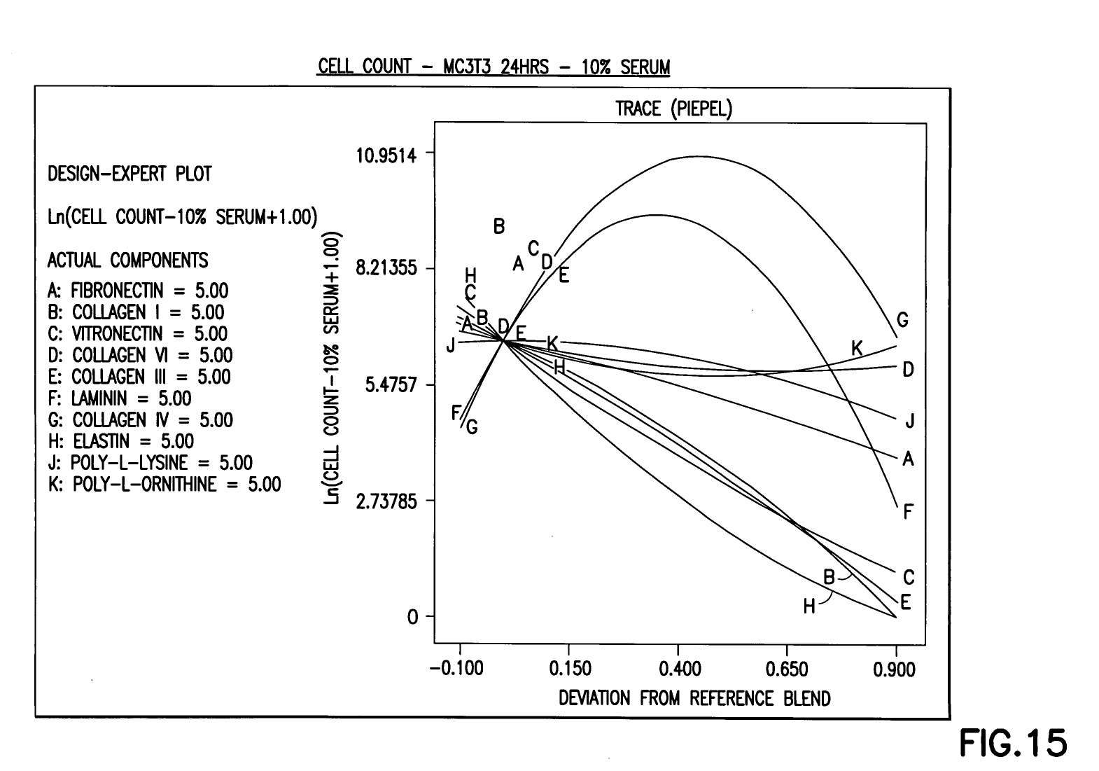

With reference now to FIG. 15, with 10% serum, any effect of poly-L-lysine that was seen in FIG. 14 diminishes, and only Collagen IV and laminin continue to show a positive effect on cell count.

It is noted that both the “best well” and “best factors” approaches are valid, but each approach can lead to different surface compositions. In the present example, the “best well” approach would lead to a surface comprising Collagen-type VI and Collagen-type III, while the “best factor” approach would lead to a surface comprising Collagen VI and laminin.

### Example 5

Use of a Statistically Designed Experiment (Plackett-Burman Design) to Screen 30 Different Agents

Design

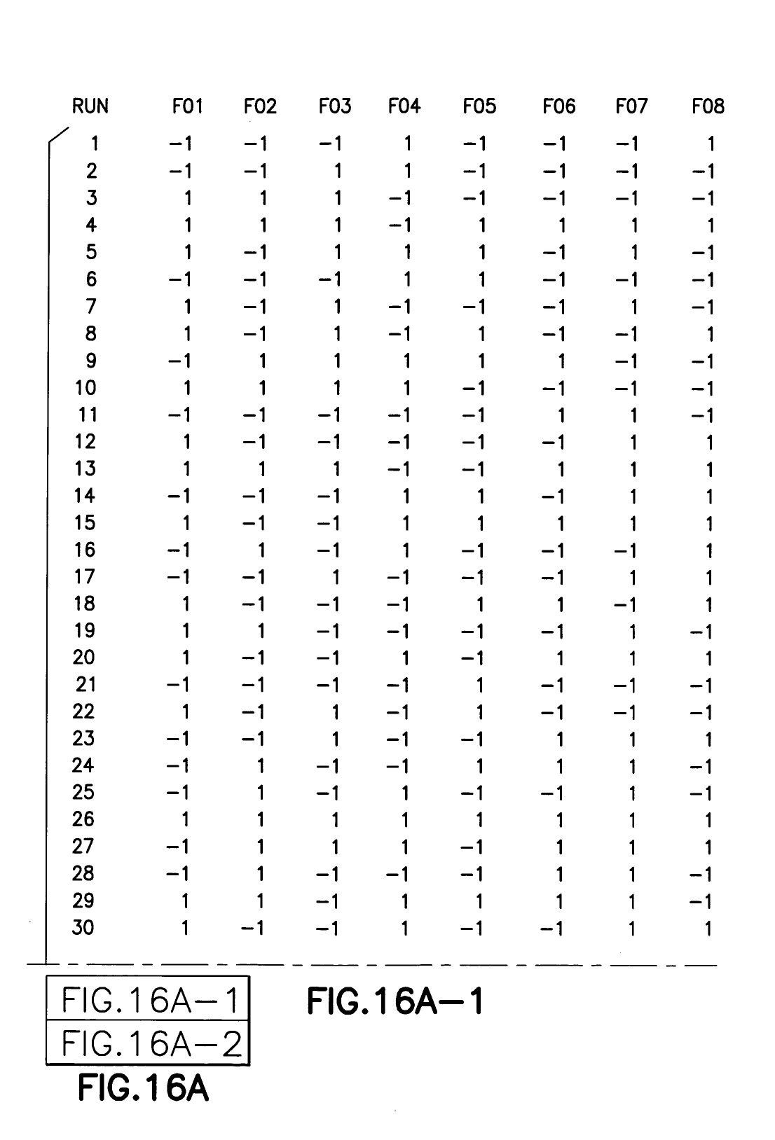

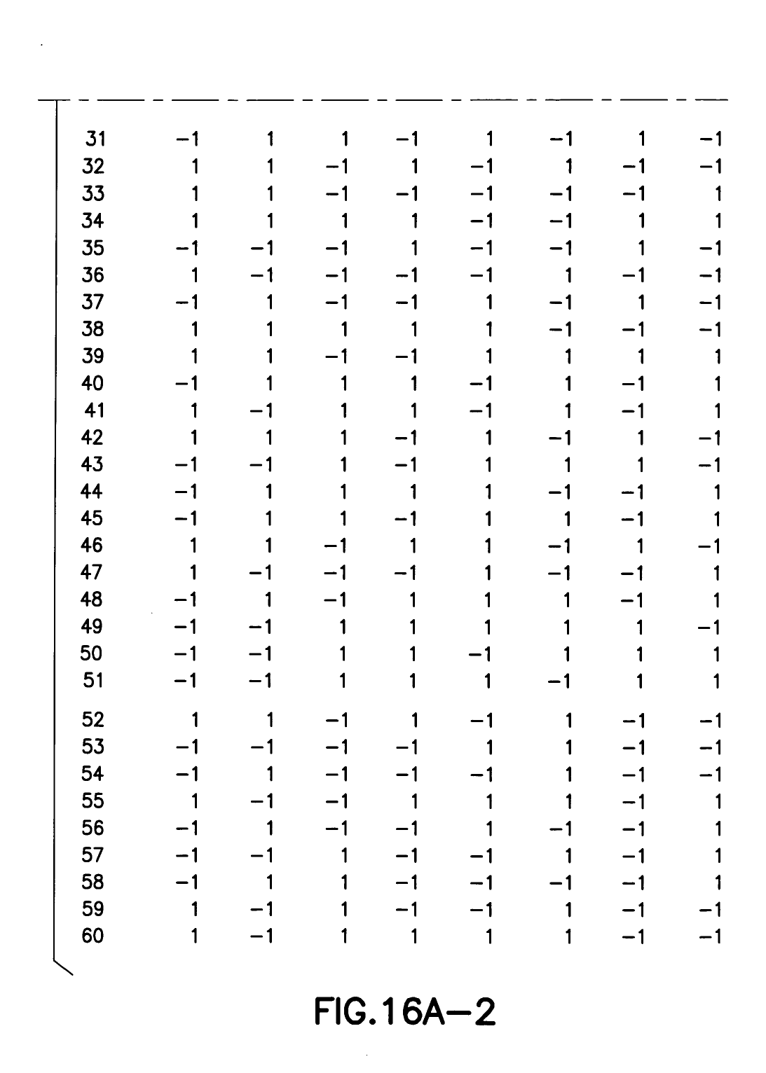

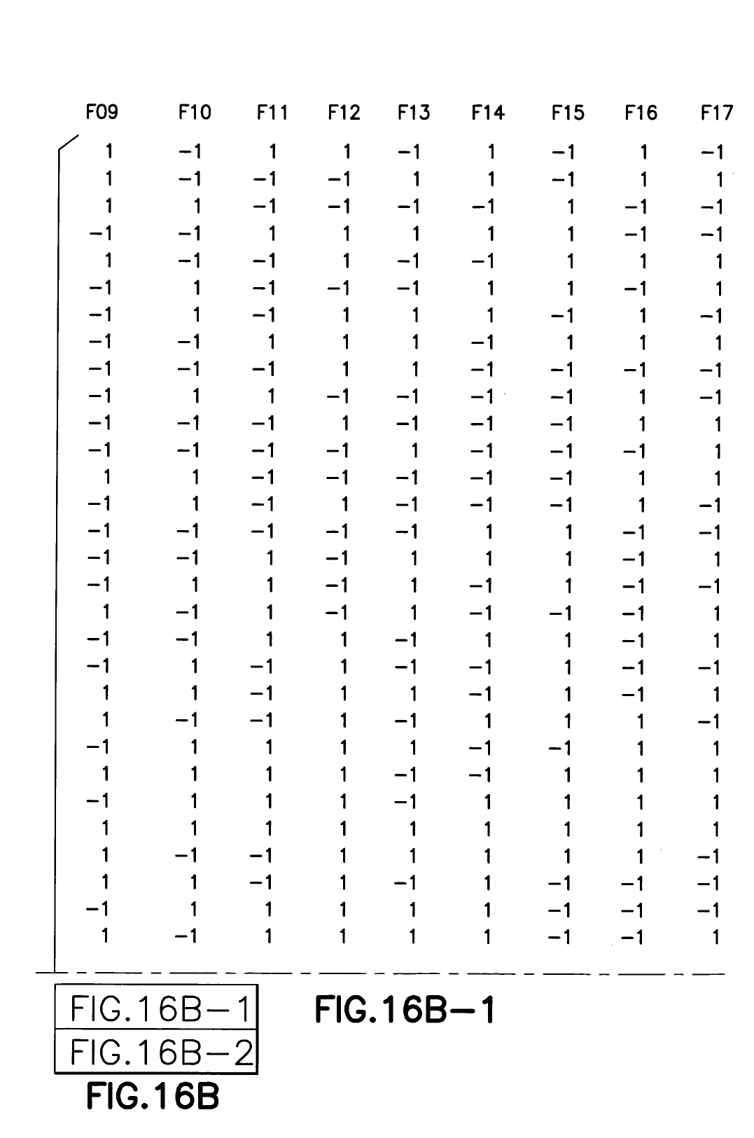

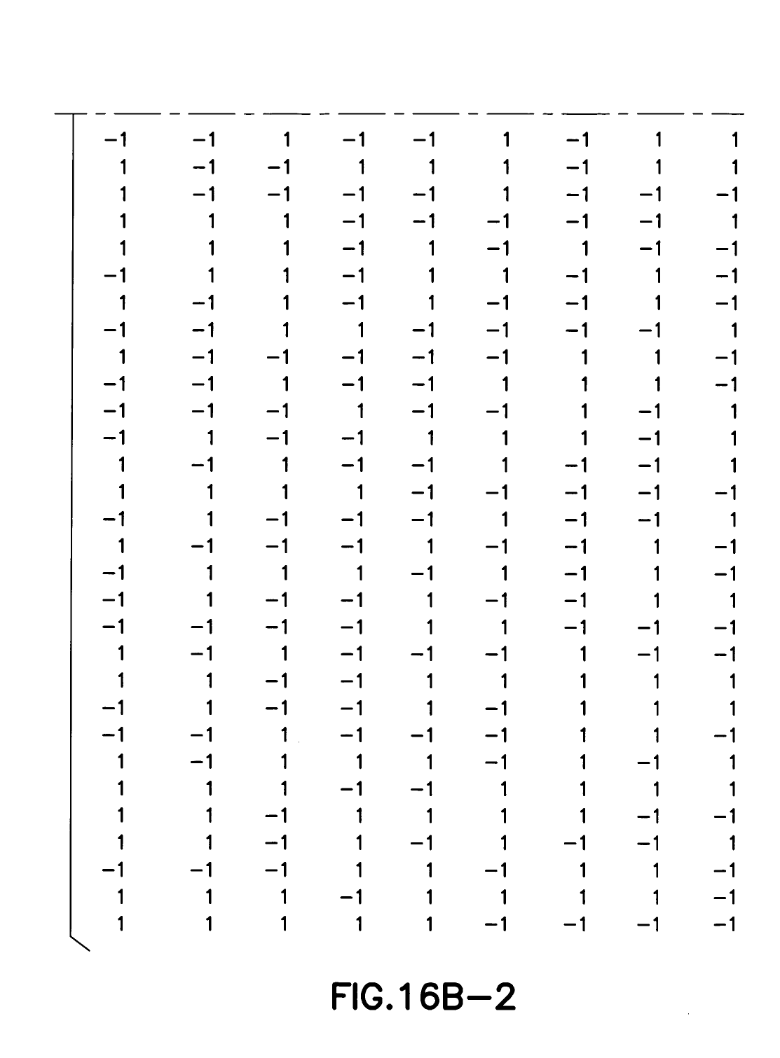

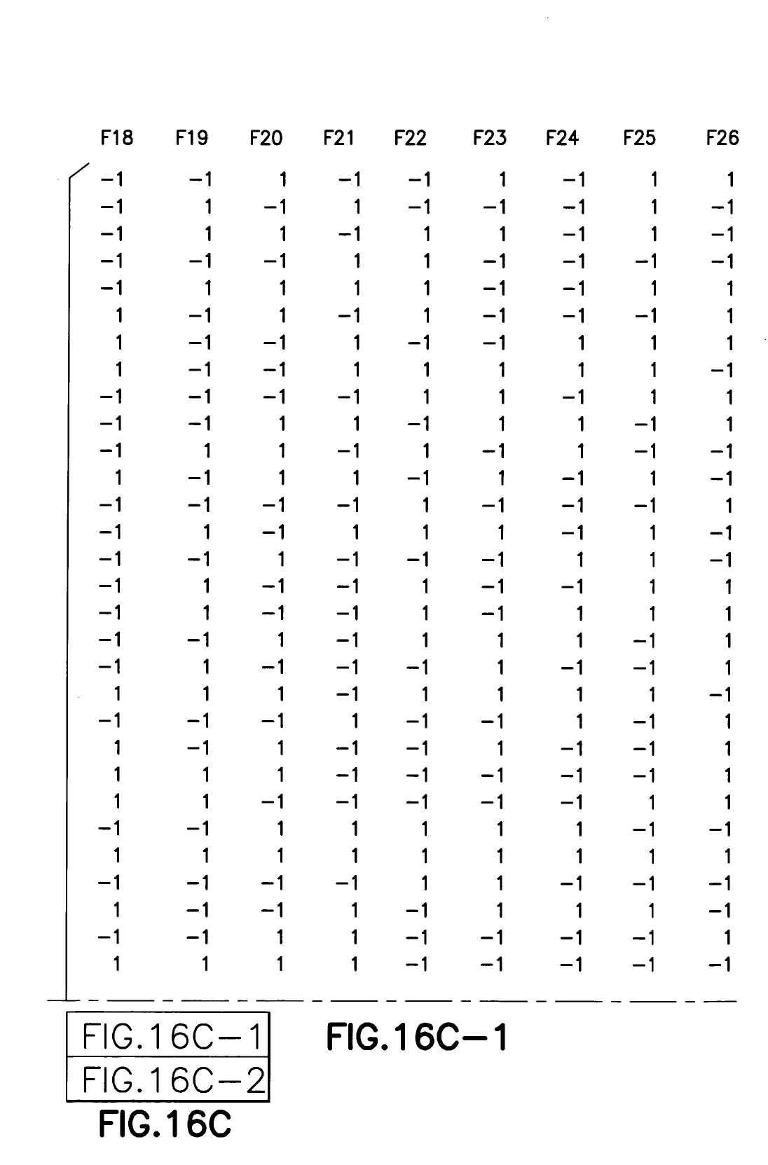

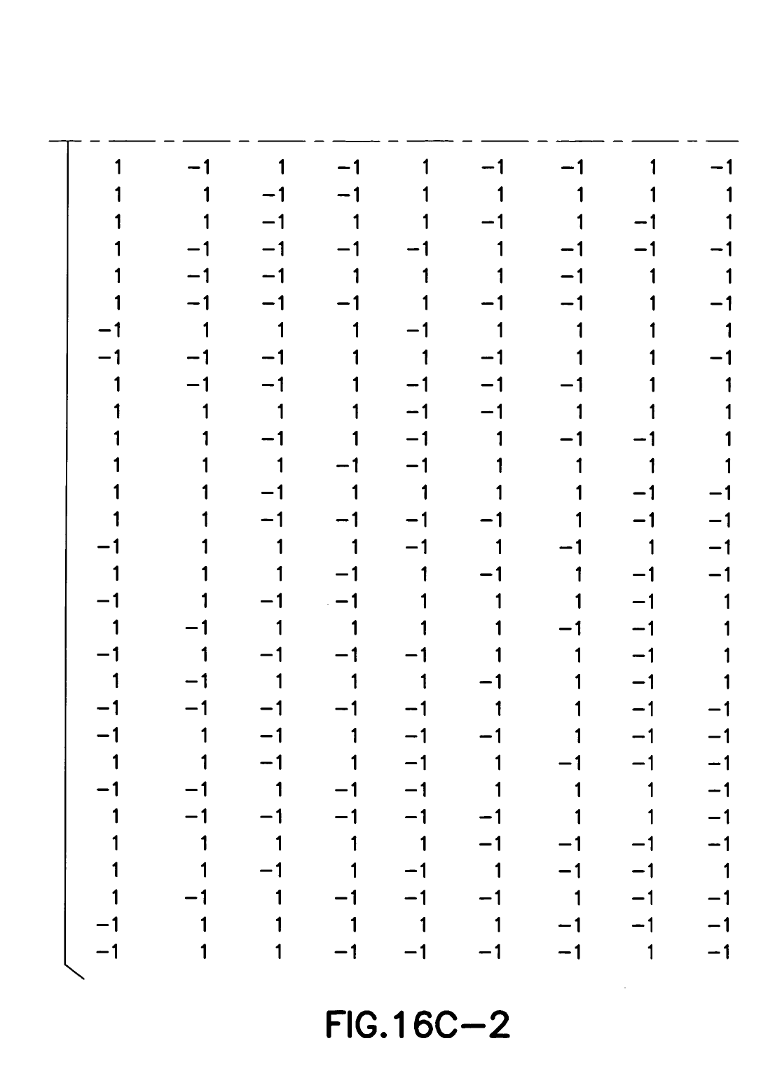

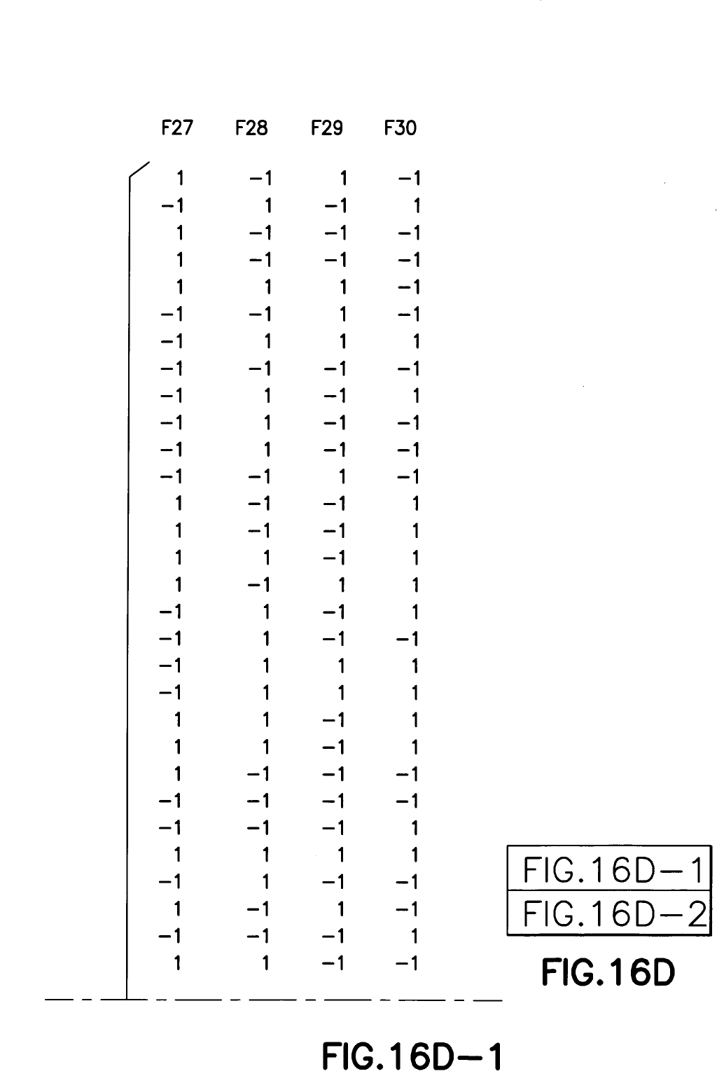

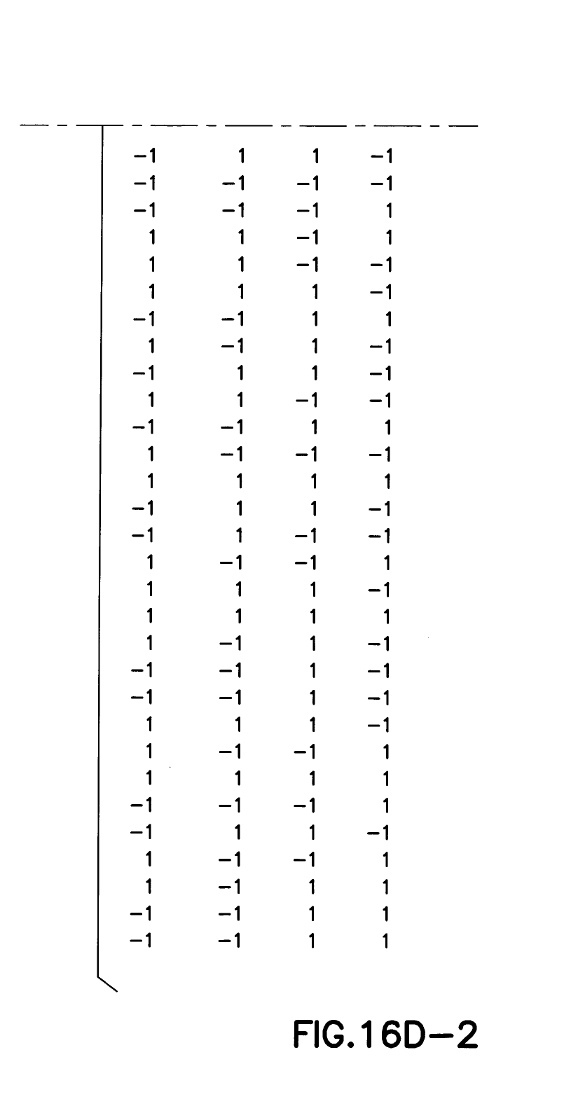

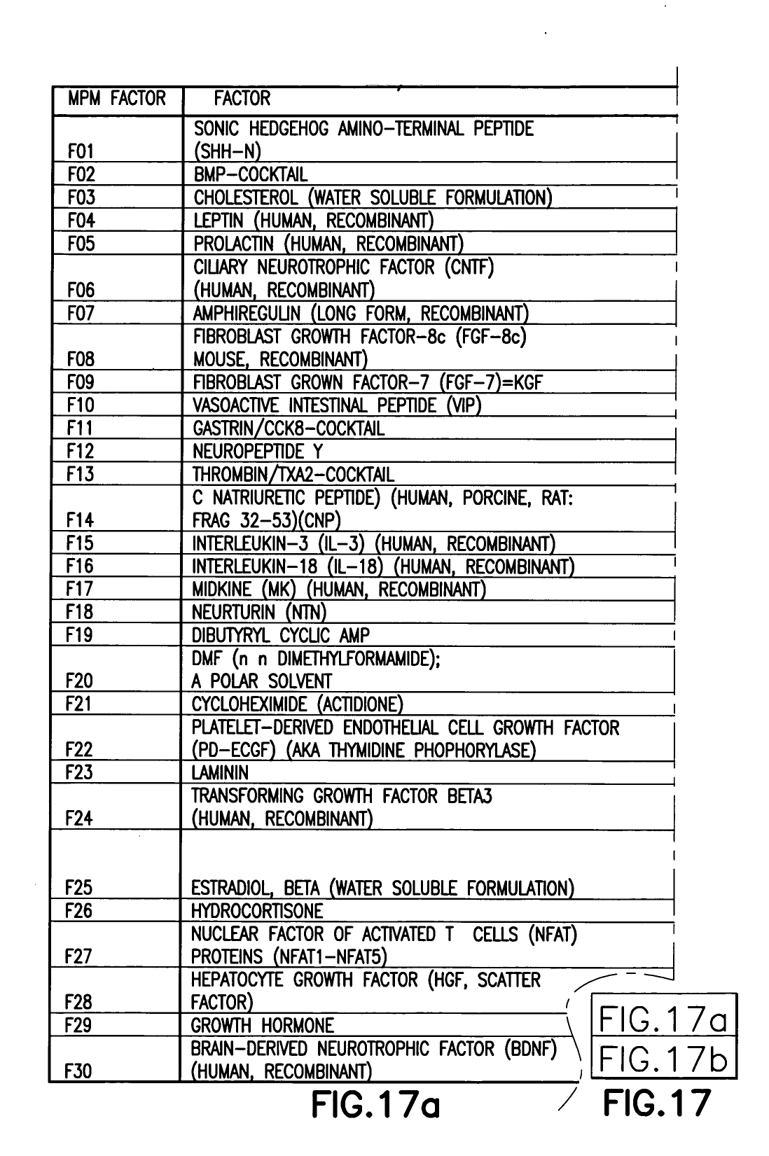

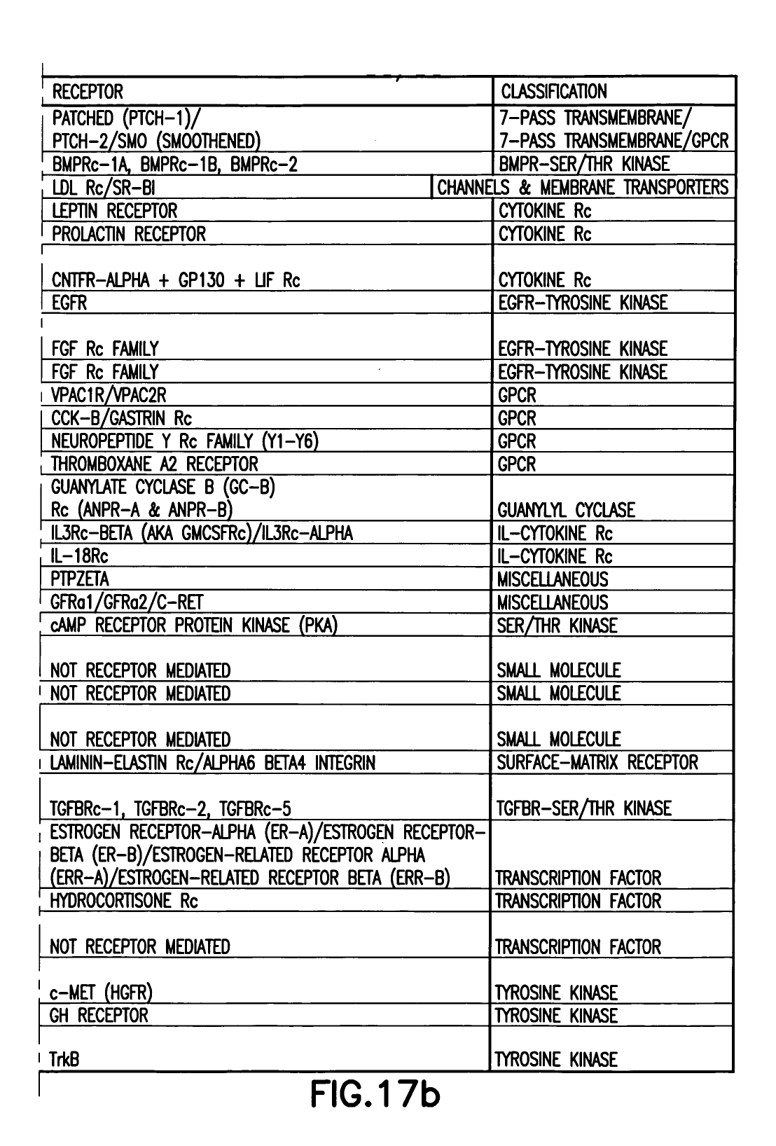

The present example describes a Plackett-Burman (PB) design as shown in FIG. 16(a-d), which was generated using a commercially available software package JMP™ from SAS Institute (Cary, N.C.). In particular, the screening design was generated using the custom design function in SAS/JMP V 4.0.5. The software package is a GUI oriented package, so there is no code to show. With reference to FIG. 16a, the first column is a list of the experimental points (runs) that translate into single wells in the 96-well plate, e.g., 60 wells in this case. The numbers in the spreadsheet itself (−1 or 1) (FIGS. 16a-d) is an indication of the level of a factor. In this example, “1” indicates the presence of the factor and “−1” indicates the absence of a factor. Moreover, in this example, if a factor is present in a given well, it is always at the same concentration in regard to the total volume of the well. The total concentration of agents may vary from well to well based on the number of agents included in the corresponding experimental run. The generic factor names are provided in the top row of FIGS. 16a-d. FIG. 17 shows the identity of each of generic factors F01-F30 in the present experiment. For example, experimental run 1 in the first column may represent well 1 of a 96-well plate. From the statistical design shown in FIG. 16(a-d), it can be seen that the following factors are present (i.e., level “1” ) in well 1: F04, F08, F09, F11, F12, F14, F16, F20, F23, F25, F26, F27, and F29.

Proposed Acquisition of Data and Statistical Analysis

Cells are plated into the wells of a 96-well plate in accordance with the design shown in the spreadsheet of FIG. 16(a-d). The seeding density is about 10,000 cells per well. Cells are incubated on the plates overnight at 37° C. The following day, media and any cells not adhering to the immobilized agents on the well surfaces are removed and any adhered cells are fixed by exposure to formalin for 15 minutes. The nuclei of the fixed adhered cells are fluorescently labeled and images are acquired with a fluorescent microscope as described above in Example 4. An image analysis software package (Meta Morph, Universal Imaging Corporation) is used to enumerate the fluorescently labeled cell nuclei and the nuclei count results for the cells are obtained. Based on these results, wells with the best experimental outcome (e.g., highest number of cell nuclei) are chosen for further optimization. By examining the contents of the wells that give the best results, information is gained regarding which factors and/or factor groups yields beneficial effects. By including many factors in the design, potentially more complex interactions between the factors can be determined. Follow-up screening experiments can focus on a particularly interesting factor combination discovered in the first round of screening.

Following the first screen, the main effects are estimated and reviewed. By “main effects”, it is meant the effect of a single agent acting independently. Interaction effects mean the combined effects of more than one single agent when the agents act in concert (not independently). At this point, relevant interactions among the agents typically are not estimated in the statistical model, but interactions among the agents would be expected to result in the best experimental runs, i.e., best wells. After the first round of screening, the best wells and the factors that are included in these wells (level=“1”) are identified. Follow-up experiments can be performed for each best well using all the factors included in the well, whether or not they had a positive, neutral, or negative effect in the preliminary statistical analysis. The experiments can be repeated with a subset of the agents identified in the best well so as to arrive at an optimum subset of factors for producing a desired response in a cell. Moreover, the experiment can be repeated, wherein the concentration of the agents in a best well are varied. Follow-up experiments can also be performed with the subset of single agents that had statistically significant main effects or by combining a subset of the best single agents with a subset identified in the best mixtures.

It has been proposed that the control of cellular phenotypes via extracellular conditions is governed by high order interactions among the factors in the extracellular environment. The Plackett-Burman design presented here is believed to provide good statistical estimates of the main effects and also provides the opportunity to observe a diverse set of combinations of factors among its experimental runs. In this case, higher-order interactions would be expected to result in specific experimental runs being “best wells” over and above what could be predicted by the individual main effects of the agents in the best wells.

1. An automated method for identifying agents that cause a phenotypic change in a cell comprising the steps of:
providing receptacles in an array;
providing a statistical design including generic factor names, factor levels, and experimental runs;
utilizing a software program to generate a computer representation of said statistical design, said computer representation being generated by automatically mapping the identities of agents to said generic factor names, by mapping the concentration or amounts of said agents to said factor levels, and by mapping the locations of said receptacles within said array to said experimental runs;
placing different mixtures of single said agents into select ones of said receptacles in said array according to said computer representation of said statistical design;
contacting said placed mixtures with said cells;
acquiring data indicative of a phenotypic change in said contacted cells;
utilizing a processor including an algorithm for comparing said phenotypic data with said statistical design to identify which of said mixtures of single agents and/or or which of said single agents in said mixtures are effective in causing said phenotypic change in said contacted cells; and
storing said statistical design, said agent identities, said computer representation of said statistical design, said acquired data, and the results of said algorithm comparison in one or more databases.

2. The method of claim 1, wherein a user inputs said identities and said concentrations or amounts of said agents into said software program.

3. The method of claim 1, wherein a user inputs said statistical design into said software program.

4. The method of claim 1, further comprising the step of generating a computer program for a robotic system to perform said placing step.

5. The method of claim 1, further comprising the step of placing single said agents into others of said receptacles in said array.

6. The method of claim 1, wherein said receptacles includes a surface which is coated with an agent-immobilizing material.

7. The method of claim 6, further comprising the step of covalently immobilizing said mixtures of single agents to said agent-immobilizing material on said receptacle surface.

8. The method of claim 6, wherein said agent-immobilizing material is a biocompatible polymer that includes reactive groups for covalently immobilizing said agents.

9. The method of claim 1, wherein the all of said databases are a single integrated or federated database.

10. The method of claim 1, wherein the identification of said mixtures that are effective in causing said phenotypic change is determined by fitting statistical models

11. The method of claim 1, wherein the identification of said mixtures that are effective in causing said phenotypic change is determined by direct comparisons among said mixtures and/or against controls.

12. The method of claim 1, wherein said processor further includes an algorithm for comparing the performance of said single agents or said mixtures of said single agents over multiple experiments in order determine trends or patterns, wherein said comparisons are stored in a database and can be periodically updated.

13. The method of claim 1, wherein said statistical design is a fractional factorial design, a d-optimal design, a mixture design, or a Plackett-Burman design.

14. The method of claim 1, wherein said statistical design is a space-filling design based a coverage criteria, a lattice design, or a latin square design.

15. The method of claim 1, wherein said agents comprise cellular ligands and/or extrinsic factors.

16. The method of claim 15, wherein said agents are selected from the group consisting of extracellular matrix proteins, extracellular matrix protein fragments, peptides, growth factors, cytokines and combinations thereof.

17. The method of claim 1, further comprising repeating said steps with a subset of said identified mixture of single agents.

18. The method of claim 1, further comprising repeating said steps, wherein the concentrations of single agents in said identified mixture of single agents are varied.

19. The method of claim 1, further comprising the step of identifying internal cellular mechanisms associated with said phenotypic change.

20. The method of claim 19, wherein said identifying of cellular mechanisms comprises extracting scientific information on cellular pathways and comparing said extracted information with said identified mixture of single agents and said phenotypic change.

21. The method of claim 20, wherein said information is computer-extracted.

22. The method of claim 20, wherein said information comprises gene expression data, protein expression data, cellular phenotype data, signal transduction data, data on cellular pathways, and combinations thereof.

23. The method of claim 19, wherein said identifying of cellular mechanisms comprises identifying genes and/or proteins expressed by said cells in the presence of said identified mixture of single agents.

24. The method of claim 19, wherein said identifying of cellular mechanisms comprises identifying which receptors on said cells are activated in the presence of said identified mixture of single agents.

25. The method of claim 1, wherein said processor further includes a first application program for calculating the likelihood that a cellular pathway, protein, or gene is involved in changes in cellular phenotype associated with said identified mixture of single agents, wherein said cellular pathway or protein is determined using scientific information.

26. The method of claim 25, wherein said scientific information is selected from the group consisting of gene expression data, protein expression data, cellular phenotype data, signal transduction data, data on cellular pathways, and combinations thereof.

27. The method of claim 25, wherein said scientific information is stored in one or more databases.

28. The method of claim 25, wherein said scientific information comprises the identification of genes and/or proteins expressed by said cells in the presence of said identified mixture of single agents.

29. The method of claim 25, wherein said scientific information comprises the identification of receptors on said cells which are activated in the presence of said identified mixture of single agents.

30. The method of claim 1, wherein said phenotypic data is acquired by inmunocytochemistry analysis.

31. The method of claim 30, wherein said immunocytochemistry analysis determines whether biological markers are present that indicate proliferation and/or differentiation of said cells in the presence of a particular mixture of single agents.

32. A system for identifying agents that cause a phenotypic change in a cell, comprising:
an array of receptacles, selective ones of which are for receiving (i) different mixtures of single said agents, and (ii) fluid including said cells;
a statistical design including generic factor names, factor levels, and experimental runs;
a software program for generating a computer representation of said statistical design, wherein said software program automatically maps the identities of said agents to said generic factor names, maps the concentration of or amounts of said agents to said factor levels, and maps the locations of said receptacles in said array to said experimental runs;
acquired experimental data indicative of said phenotypic change in said cells;
a processor including an algorithm for comparing said experimental data with said statistical design to identify which of said mixtures of single agents and/or which of said single agents in said mixtures are effective in causing said phenotypic change in said cells; and
one or more databases for storing said statistical design, said agent identities, said computer representation of said statistical design, said acquired experimental data, and the results of said algorithm comparison.

33. The system of claim 32, wherein said databases are a single integrated or federated database.

34. The system of claim 32, further comprising a robotic system to place said mixtures of single agents correctly in said receptacles based on said computer representation of said statistical design.

35. The system of claim 34, further comprising a computer program with instructions for said robotic system to place said mixtures of single agents correctly in said receptacles based on said computer representation of said statistical design.

36. The system of claim 31, wherein said processor further includes an algorithm for comparing the performance of said single agents or said mixtures of said single agents over multiple experiments in order to determine trends or patterns, wherein said comparisons are stored in a database.
